(19) United States
(12) Patent Application Publication
(10) Pub. No.: US 2018/0036531 A1
(43) Pub. Date: Feb. 8, 2018

(54) DEVICE, SYSTEM AND METHOD FOR THE TRANSMISSION OF STIMULI

(71) Applicant: WEARABLE LIFE SCIENCE GMBH, Nürnberg (DE)

(72) Inventors: Philipp G. SCHWARZ, Frankfurt (DE); Nordin KOUACHE, Frankfurt (DE); Kay RATHSCHLAG, Weilburg (DE); Shahid MEHBOOB, Frankfurt (DE); Patrick THUMM, Villingen-Schwenningen (DE)

(73) Assignee: Wearable Life Science GmbH, Numberg (DE)

(21) Appl. No.: 15/551,958

(22) PCT Filed: Feb. 18, 2016
(86) PCT No.: PCT/EP2016/053490

§ 371 Date: Aug. 18, 2017

(30) Foreign Application Priority Data
Feb. 18, 2015 (DE) 20 2015 001 313.9
Feb. 27, 2015 (DE) 10 2015 002 484.1
Feb. 27, 2015 (DE) 10 2015 002 565.1
Aug. 14, 2015 (DE) 20 2015 005 645.8
Feb. 12, 2016 (EP) PCT/EP2016/000235

(51) Int. Cl. A61N 1/36; A61B 5/0488; G06F 3/01; G01S 19/19; G06F 19/00; A61N 1/04; A61B 5/0205

(52) U.S. Cl. CPC A61N 1/36003; A61N 1/0484; A61N 1/36031; A61B 5/0488; A61B 5/02055; A61N 1/0452; G01S 19/19; G06F 19/3481; G06F 3/015; G06F 3/016; A61B 5/053

(57) ABSTRACT

A device and to a system and a method for transmitting stimuli to a user. The stimuli can include stimuli caused by electrical muscle stimulation or haptic stimuli such as vibrations. The system simplifies the use of the corresponding stimuli inter alia in that parameters can be measured during the use and the type and specificity of the stimuli can be changed depending on the measured parameters. The systems, devices and methods are particularly suitable for use in sports.

### FIELD OF THE INVENTION

The invention relates to a method and an apparatus for receiving electrical signals from a body and for transferring electrical signals to a body. The present invention relates, in particular, to a system for controlling stimulation pulses. The prior art has disclosed stimulation pulses, in particular electrical muscle stimulation (EMS) for stimulating various biological tissues such as muscles and nerves.

In particular, the invention relates to an apparatus, or a system, and a method for transmitting stimuli to a user. The stimuli may comprise electric muscle stimulation stimuli or else haptic stimuli such as vibrations. The system simplifies the use of the corresponding stimuli, inter alia by virtue of parameters being able to be measured during the use and the type and characteristics of the stimuli being modifiable depending on the measured parameters. The systems, apparatuses, and methods are particularly suitable for use in sports.

### BACKGROUND OF THE INVENTION

Electrical muscle stimulation finds particular application in the case of specific indications in the medical field, such as preferably medical rehabilitation, and in sports, in particular high-performance sports, and in the fitness sector. EMS training brings about its positive effects largely by way of neuronal improvements, such as e.g. an increased activatability. A person who has trained with EMS has an increased muscle mass, which, for example, reduces the frequency of falls, or the consequences thereof, in the elderly.

Document CA 2537177 A1 discloses an apparatus for muscle stimulation, provided to assist the cardiac pumping function by way of muscle stimulation. The apparatus comprises a pulse-producing unit for producing and outputting an electrical stimulation pulse, a control unit for controlling the pulse-producing unit and for ensuring that the stimulation pulse reaches the muscle to be stimulated. A provided determination unit serves to determine an average stimulation frequency within a definable period of time, and also pulse storage means with a computing unit. A computing unit serves to calculate a stimulation pattern.

There is a need for a system for pulse stimulation which has improved user-friendliness and improved operational friendliness such that a user can use such a system without external help. Here, the system should specifically cater to the concrete demands of training with EMS and impart fun to the person training, as a result of which the person training increases the use duration and thus arrives at improved results. Moreover, the emission of the stimulation pulses is often complicated in the prior art as a result of predeterminable intensity and duration or as a result of predeterminable exercise programs, and an individualized adaptation to the specific conditions of the user, in particular during use, is only permitted to a small extent.

The underlying problem is based on the complaints by athletes and rehabilitating persons about the use of EMG devices (electromyography: electrophysiological method in neurological diagnostics), in which the electric muscle activity is measured, and EMS devices, in which electrical muscle stimulation (EMS) is transferred not by way of the electrical pulses from the brain but externally by low stimuli currents. Here, electrical pulses are transmitted to muscle groups by way of electrodes that have been worked into functional apparel, as a result of which the muscle or muscles undergo a contraction. In most systems, this is a stationary unit which can only receive EMG signals or only transmit EMS signals. Also, no individual parameters are captured. There is no individual adaptation to the physical strain in the existing systems.

By way of the existing devices, it is not possible to carry out an adaptation of transmitted signals on the basis of received signals.

Further aspects emerge from the application of electrical signals within the scope of a virtual world (virtual reality, VR). Current applications render it possible to move in a virtual world, for example by using specific goggles or screen presentations, but there is no feedback between the user and the virtual world. In particular, there is no real interchange between avatar (e.g. virtual trainer) and user. The terms “virtual reality” and “virtual world” are used synonymously in this application.

### SUMMARY OF THE INVENTION

It is an object of the present invention to provide a system which overcomes the aforementioned disadvantages. It is a further object of the present invention to provide for controlling stimulation impulses for recreation, in particular non-sports-related recreation, in particular the computer games sector.

There is a need for a system for pulse stimulation which has improved user-friendliness and improved operational friendliness such that a user can use such a system without external help. Here, the system should specifically cater to the concrete demands of training with EMS and impart fun to the person training, as a result of which the person training increases the use duration and thus arrives at improved results. Moreover, the emission of the stimulation pulses is often complicated in the prior art as a result of predeterminable intensity and duration or as a result of predeterminable exercise programs, and an individualized adaptation to the specific conditions of the user, in particular during use, is only permitted to a small extent.

It is an object of the present invention to provide a system which overcomes the aforementioned disadvantages. It is a further object of the present invention to provide for controlling stimulation impulses for recreation, in particular non-sports-related recreation, in particular the computer games sector.

The invention is based on the object of developing a device that can be used in multifaceted ways and renders it possible to individually match muscle activity signals and muscle stimulation signals in order to train a human. The method allows athletes to learn, train or improve ideal sequences of movement.

These objects of the invention are achieved by the subject matter of the main claim. Preferred embodiments emerge from the dependent claims. Further, this description discloses a multiplicity of further embodiments which, on their own or in combination with the variants described herein, contribute to solving the problem or solving further partial problems.

The terms “system” and “apparatus” are used synonymously in this description. Within the scope of this document, the term “sensor” should be interpreted broadly. Thus, an electrode which is suitable for EMS transfer is also a sensor within this meaning since, depending on the circuit, data may also be measured. By way of example, the skin transfer resistance or capacitances can be measured by way of the electrode. In this description, all embodiments which have electrical muscle stimulation stimuli as subject matter also comprise other tactile stimuli, such as haptic stimuli and, in particular, vibration stimuli as well. However, preferably, the embodiments described herein relate to electrical muscle stimulation and the transferred stimuli are preferably electrical muscle stimulation stimuli, even if individual embodiments refer to haptic stimuli or tactile stimuli. All aspects described herein which relate to a configuration of the apparatus or of the system and express that the apparatus or system is embodied, configured or otherwise suited to carry out a specific method step should be understood in such a way that these method steps also represent possible and equally preferred steps of the method according to the invention found herein.

The muscle activity signals (EMG) are received by way of one and/or more electrodes. These may be worked into a textile. The electrodes are able to communicate in a wired and/or wireless manner. They can be addressed by way of a control unit, transmit signals to the control unit or receive said signals. The control unit may be a mobile terminal, such as, in particular, a smartphone, which communicates in a wireless and/or else wired manner with the system. Moreover, by way of suitable shaping, the system may be integrated in any conceivable apparel piece (e.g. suit, jacket, trousers, socks, underwear, hat, shirt). Moreover, the system may be integrated into shoes, gloves, and all materials which can be worn on a body.

The apparatus according to the invention is embodied to be worn on the human body, comprising fastening means which place the apparatus directly on a body part. The apparatus has touching contact with the user, in particular by way of sensors or electrodes. An apparatus unit may consist of a receiving sensor and/or transmitting sensor (EMG and/or EMS). These sensors may be worked into a textile, individually and/or a plurality together, or else be positioned on the body by fastening means. The sensors may also be processed in the form of a yarn, or else as individual electrodes. The individual electrodes and/or the plurality of electrodes may be connected by way of worked-in pathways and/or may be connected wirelessly. The EMG signals can be transmitted to a control device via radio and/or wired signals, the EMS sensors can receive signals via radio and/or in a wired manner, in order to stimulate the muscle.

Here, specific information items are preferably prepared on a mobile terminal (e.g. a smartphone) or else on a stationary terminal (e.g. a PC), said information items then being transferred to the apparatus according to the invention. In this case, the apparatus according to the invention preferably has an appropriate processor and main memory, and optionally also one or more of the following sensors: BIA (bio impedance analysis) sensor, an ultrasonic sensor, EMG sensor, EMS sensor, movement sensor, NIRS (near-infrared) sensor, magnetoresistance sensor, moisture sensor, ECG sensor (in particular for an HRV measurement), strain gauge (in particular for measuring the respiratory rate), lactate sensor, temperature sensor, blood sugar sensor and contact sensor. All sensors may be worked into the textile and/or into the control device, which may be fastened to the apparel. The sensors may be actuated in a wired and/or wireless manner. The control unit can operate offline, when it is in use. Alternatively, the user can select between the two modes of online/offline. The wireless receiver is embodied to receive or transmit instructions.

As already mentioned above, the fastening means may be integrated into any apparel piece (suits, jackets, trousers, skirts, dresses, socks, underwear, hats, tops, cuffs), work apparel (e.g. of the fire department, of the police, of the military), sports apparel (e.g. diving suit, wet suit), survival suits, functional clothes, or in any conceivable apparel and/or any conceivable apparel piece. Moreover, the system may be integrated into shoes, gloves, belts and all materials which can be worn on a body.

In a further embodiment, the apparatus comprises a plurality of sensors at different spatially spaced-apart positions within the textile or the apparel piece, in particular in such a way that the different position of a body part may be captured.

In a further embodiment, the system may have electronics that are configured to control one or more channels in order to stimulate one or more muscles. The control is preferably brought about by way of a microcontroller which renders it possible to control the sensors by way of one or more channels. For the purposes of providing further information items for distinguishing the information items, the electronics may produce different stimuli patterns which are actuatable externally, comprising one and/or more of the following parameters: intensity, frequency, time duration, time interval, signal sequence.

The intensity may be the strength of the electrical signal. The frequency may be the repetition of the electrical signal or the frequency of the pulse itself. The time duration can relate to the length of the electrical signal. The time interval can relate to the spacing between the individual pulse intervals. The signal sequence can be a slow rise in the electrical signal or a specific pattern of electrical signals.

In a further embodiment, the system may be used, in particular in the case of long flights, for thrombosis prophylaxis by virtue of individual muscles in the lower extremities being stimulated during the flight. To this end, the electrodes can be worked into the stockings or cuffs of the user.

In a further embodiment, the electrodes consist of a conductive yarn which is surrounded by a titanium layer; the latter is preferably a few atoms thick.

In another embodiment, electrodes may also be worked into a textile as individual zones, for example for the upper body (or individual extremities) and/or for the lower body (or individual extremities), wherein the zones consist of individual components or a plurality of components, which are current carrying or not current carrying.

In a further embodiment, the electrodes may consist of a material that requires moisture in order to transfer the pulses to the skin; these electrodes are combined with and/or folded, warp-knitted, embroidered or weft-knitted into a hydrophilic yarn. Alternatively, the electrodes may be provided with a moisture-providing layer that lies between the skin and conductor.

In a further embodiment, the electrodes may be manufactured from a conductive polymer, e.g. silicone. The surface of the electrode is brought into the corresponding shape preferably by way of a suitable mold when extruding/molding or within another production method, in which the polymer, in particular silicone, is brought into the corresponding shape, such that no smooth surface arises. It is preferred for the polymer to be able to be provided with an uneven surface in order to ensure an ideal adaptation to the anatomy of the body. These polymer electrodes, in particular silicone electrodes, may be multilayered, i.e. consist of a nonconductive layer and a conductive layer. In order to avoid tear propagation, the electrodes may contain an integrated fabric ply which, at best, is preferably just as stretchable as the conductive silicone.

In a further embodiment, the textile or the apparel piece may be manufactured from conductive material. This additionally ensures that the current supplied is uniformly distributed in the electrode. Additional contact pressure can be produced by a cushion between the electrode and the outer layer, in particular in the case of concave body regions, for example between the breasts. A special form of the electrode may consist either of two outer ring electrodes and an inner ring electrode or of an outer and inner circle (the circles are each configured as an electrode). In addition to the use with bipolar currents, these electrodes are also suitable for a unipolar current.

Here, the current should enter the body via the larger electrode in each case. The textiles into which the electrodes have been worked preferably have a partial compression and/or different compression zones. The conductor tracks, which are preferably embroidered on, are preferably guided over the textile in a bendable and/or elastic manner.

In another embodiment, a unit that is connected to the stimulation unit and preferably elucidates the stimulation at the hand or the wrist, can preferably be provided via a cable and/or in a wireless manner. In particular, this unit can preferably signal the change between pulse and pause. This may be brought about by acoustic signals and/or visual signals and/or haptic signals, in particular by vibrating. This unit may also be used to control the intensity. Or else it can indicate the signals, in particular by means of LEDs. This unit is preferably fastened to the user by way of a band, which is not a wristband but instead worn on the back of the hand. Preferably, it is additionally fastened to the thumb by means of a loop.

A further part of the invention is a training method and/or monitoring method using an apparatus as described above, wherein training information items and/or monitoring information items for a user are forwarded as tactile stimuli to the user and/or received by said user.

A further part of the invention is a suit, worked into which are sensors which analyze local muscle activity by way of EMG signal measurements and compare these to the contralateral side. If hyperactivity is recognized, the muscle may be stimulated on the contralateral side in order to trigger an antagonistic inhibition in the tensioned muscle so as to relax it.

Below, further conceivable and preferred configurations of this invention are intended to be described, these representing preferred systems and methods of this invention as alternative configurations or in combination with the further features described herein.

Encompassed by the invention is also a system comprising at least a sensor, at least a data processing unit and at least a pulse unit, wherein

a. the sensor is suitable for measuring a measurement value,

b. the data processing unit is configured to compare the measurement value to a threshold and to generate a control signal for the pulse unit when the measurement value and the threshold have a predeterminable relationship to one another,

c. the pulse unit is suitable for triggering stimulation pulses and configured to modify one or more stimulation pulse parameters depending on the control signal.

The objects described herein are also achieved by a system for controlling stimulation pulses during a stimulation on a user, comprising at least a data processing unit, which is configured to generate a control signal for a pulse unit, and a pulse unit, wherein the pulse unit is suitable for triggering stimulation pulses, and wherein the pulse unit comprises at least a channel, wherein at least two electrodes are connectable to the channel and controllable independently of one another, wherein the system is preferably a system as described above.

The objects described herein are also achieved by a method for controlling stimulation pulses during a stimulation on a user using a system, in particular a portable/wearable system, according to the present invention. Such a method, in which a pulse unit triggers one or more stimulation pulses, comprises at least the following steps:

a. measuring a measurement value,

b. comparing the measurement value to a threshold,

c. generating a control signal if the measurement value and the threshold have a predeterminable relationship to one another,

d. modifying a stimulation pulse parameter depending on the control signal.

In this context, it should be understood that any feature that is described herein in conjunction with a system according to the present invention can also be a feature of a method according to the invention, and vice versa.

Such a system according to the invention for controlling stimulation pulses and a method according to the present invention for controlling stimulation pulses during a stimulation on a user using such a system according to the invention advantageously render it possible, during a stimulation or during a stimulation application, to modify stimulation pulse parameters depending on the measurement values measured by the sensor. This firstly allows the provision of immediate feedback to the user depending on the measured measurement values; secondly, the immediate and automated adaptation of stimulation pulse parameters depending on the measurement values measured by the sensor is rendered possible.

In particular, a system and/or method according to the invention can compare, by means of suitable algorithms, the measurement value measured by means of the sensor to a threshold. Such an algorithm may advantageously be predetermined or predeterminable in the data processing unit. If it is possible on the basis of the algorithm to determine that the measurement value and the threshold have a predefined relationship to one another, an appropriate control signal is generated and a pulse parameter is modified depending on the control signal. A corresponding stimulation pulse with a modified pulse parameter can then be triggered by the pulse unit. Hence it is possible, for example, to increase or reduce the stimulation pulse intensity depending on the measurement value.

Such a system and/or method according to the present invention, and a stimulation training, in particular an EMS training, that can be carried out by means of the method and/or the system, therefore provide an improved system and method which, in relation to systems or methods known from the prior art, are improved in respect of their effect, applicability and acceptance by the user. In particular, in its various aspects, the present invention provides a more effective system or method for controlling stimulation pulses during a stimulation on a user. Here, in particular, conventionally available components, such as sensor, data processing unit, and pulse unit may be used for reaction and/or feedback to the user, and not only for calibration.

In conjunction with the present invention, the term “stimulation on a user” should preferably be understood to mean a single application of possibly a plurality of stimulation pulses on the user, for example a single medical treatment or session during which stimulation pulses are administered to the user, a training unit or the like. However, it will immediately be understood that, depending on the specific application of the method or the system according to the present invention, a repetition of the application, in particular a repetition of the method, is possible or desired. Particularly in the case of the medical or sports-related application of the system or method according to the invention, such a “stimulation on a user” may be part of a treatment or training which comprises the multiple application of the “stimulation on the user”.

The phrase “controlling stimulation pulses”, as used herein, should preferably be understood to mean that the administration of a stimulation pulse or of a plurality of stimulation pulses is controlled by the system according to the invention. Here, such control comprises, in particular, that a pulse unit is controlled in such a way that this pulse unit triggers individual stimulation pulses or a plurality of stimulation pulses which, depending on the control signal, are changeable in one or more stimulation pulse parameters. Therefore, the phrase “controlling stimulation pulses” should preferably also comprise that one or more stimulation pulses are changed individually or in combination in terms of one or more stimulation pulse parameters, depending on the control signal. A “sensor which is suitable for measuring a measurement value” should preferably be a sensor which is suitable for capturing at least a physical or chemical or position or acoustic variable. A “sensor”, as used herein, preferably refers to a detector for such a physical or chemical or position or acoustic variable, a recorder or (measurement) sensing element for such a physical or chemical or position or acoustic variable. Here, a sensor can be understood to be a technical component which can capture such a physical or chemical or position or acoustic variable qualitatively or quantitatively as the measurement value. Such a physical or chemical or position or acoustic variable may be selected, in particular, from the group comprising time, pressure, ultrasound, electric resistance, in particular electric resistance of a biological tissue, preferably of the muscles; acceleration, positioning, position, movement, pulse frequency, heart rate, temperature, thermal radiation, moisture, pressure, sound, brightness, or the like, pH value, ionic strength, electrochemical potential, material conditions of its surroundings. These physical or chemical or position or acoustic variables are captured by means of their physical or chemical or position or acoustic effects by the sensor as a measurement value and transmitted to the data processing unit, the latter being configured to compare the measurement value to a threshold and generate a control signal for the pulse unit if the measurement value and the threshold have a predefinable relationship to one another. In particular, such a physical or chemical or position or acoustic variable may be a physical or chemical or position or acoustic variable that is characteristic for the body of the user. It should be understood that, in the context of a sensor which is suitable for capturing an electric resistance as a measurement value, this refers, in particular, to an electric resistance of a biological tissue, preferably of a muscle. However, it should also be understood that, additionally or alternatively, it is also possible to measure the electric resistance of other biological tissues as a measurement value of a sensor within the scope of the present invention, in particular e.g. of bones and/or skin, fatty tissue, in particular fatty tissue over a muscle, and other tissue that react to electrical pulses. The definition of the sensor used herein does not preclude that the sensor may also be configured as an electrode and additionally carries out the functions assigned to an electrode herein.

In a further preferred embodiment of the system or method according to the invention, the sensor is selected from time sensor, in particular timepiece, pressure sensor, ultrasonic sensor, acoustic sensor, contact sensor, resistance sensor, in particular for measuring the body resistance, electromyography sensor, acceleration sensor, positioning sensor, near infrared spectroscopy (NIRS) sensor, a sensor for measuring the oxygen saturation, sensor for bioelectrical impedance analysis (BIA); sensor for measuring magnetoresistance; movement sensor, contact sensor, pulse frequency sensor, heart rate sensor, ECG sensor, temperature sensor, sensor for capturing fat burning, calorie consumption sensor, sweat sensor, location sensor, in particular GPS sensor, respiration sensor, in particular for measuring respiratory rate and/or depth of respiration, spirometry sensor, lactate sensor, blood sugar sensor, pH sensor and the like.

In particular, the system according to the invention may comprise a sensor which measures the EMG activity of the user. Such a sensor may be an electromyography device or part thereof. This advantageously renders it possible to measure EMG activity of the user and trigger a stimulation pulse, in particular an EMS pulse, which is modified in one or more stimulation pulse parameters, depending on the measurement value or control signal. By way of example, the stimulation pulse is triggered as soon as it is possible to detect a deliberate activation of the muscle in a corresponding muscle group by way of the brain by using the EMG sensor. This facilitates an accurate temporal coordination between pulse and natural contraction, exhibiting advantages in respect of coordination and functionality. Such a configuration of the system or method according to the invention may be particularly advantageous for sports, but also for the rehabilitation sector.

Additionally, or alternatively, the system according to the invention may comprise an ultrasonic sensor. This advantageously allows determining the body composition of the user and triggering a stimulation pulse, in particular an EMS pulse, which has been modified in one or more stimulation pulse parameters, depending on the measurement value or control signal. The general composition of the tissue of the user lying under the sensor can be ascertained very well using ultrasound. This firstly renders it possible to ascertain the general state of the user, e.g. a fat/muscle ratio or the like, and, secondly, renders it possible to track during movements whether a muscle migrates to different positions or whether the composition of the tissue changes as a result of the movement. By way of example, subcutaneous fatty tissue will be pushed to the side by tensing and movement and the electrode will lie almost directly on the muscle. As a result, the stimulation can be appropriately increased or reduced during the contraction.

Additionally, or alternatively, the system according to the invention may comprise a sensor for measuring the body resistance. This advantageously allows determining the body resistance of the user and triggering a stimulation pulse, in particular an EMS pulse, which has been modified in one or more stimulation pulse parameters, depending on the measurement value or control signal. By way of example, this may facilitate an automatic adaptation of stimulation pulse parameters, in particular the stimulation pulse intensity, to the respective body resistance. This is advantageous, in particular, since only the overall intensity can still be adapted. The general composition of the tissue of the user lying under the sensor can be ascertained very well using the body resistance measurement. This firstly renders it possible to ascertain the general state of the user, e.g. a fat/muscle ratio or the like, and, secondly, renders it possible to track during movements whether a muscle migrates to different positions or whether the composition of the tissue changes as a result of the movement. By way of example, subcutaneous fatty tissue will be pushed to the side by tensing and movement and the electrode will lie almost directly on the muscle. As a result, the stimulation can be appropriately increased or reduced during the contraction.

Additionally, or alternatively, the system according to the invention may comprise a pressure sensor. This advantageously allows determining a pressure of the user and triggering a stimulation pulse, in particular an EMS pulse, which has been modified in one or more stimulation pulse parameters, depending on the measurement value or control signal. By way of example, such a pressure sensor may be arranged in or on a shoe of the user. An increase in pressure on the shoe sole can be measured as a measurement value and can trigger a stimulation pulse, in particular an EMS pulse, which has been modified in one or more stimulation pulse parameters, depending on the measurement value or control signal. By way of example, a stimulation pulse may be produced in accordance with the system and/or method according to the invention if the measurement value renders it clear that there is floor contact with the foot at a given time. As soon as the measurement value has sunk to a clear rest value, leaving the ground is indicated, and the stimulation pulse can be modified, in particular terminated. This, once again, facilitates an ideal functional stimulation.

Additionally, or alternatively, the system according to the invention may comprise an acceleration sensor. This advantageously allows determining an acceleration of the user, in particular a movement, and triggering a stimulation pulse, in particular an EMS pulse, which has been modified in one or more stimulation pulse parameters, depending on the measurement value or control signal. By way of example, such an acceleration sensor can measure assuming and/or exceeding certain distances from the sensor. Assuming and/or exceeding certain distances from the sensor can be measured as a measurement value and trigger a stimulation pulse, in particular an EMS pulse, which has been modified in one or more stimulation pulse parameters, depending on the measurement value or control signal.

Additionally, or alternatively, the system according to the invention may comprise a near infrared spectroscopy (NIRS) sensor. This advantageously allows determining an oxygen saturation of biological tissue, in particular the muscles, of the user, and triggering a stimulation pulse, in particular an EMS pulse, which has been modified in one or more stimulation pulse parameters, depending on the measurement value or control signal. By way of example, such a near infrared spectroscopy (NIRS) sensor can measure the oxygen saturation of biological tissue and trigger a stimulation pulse, in particular an EMS pulse, which has been modified in one or more stimulation pulse parameters, in particular the intensity, depending on the measurement value or control signal. Such an embodiment of the present invention can find advantageous use, in particular, in the sports-for-seniors sector. Such a near infrared spectroscopy sensor may be configured in such a way that it measures the oxygen saturation in the muscles of the user during a stimulation application by means of infrared light. In the process, it is possible, in particular, to measure the proportion, in percent, of the hemoglobin and myoglobin carrying oxygen in the capillaries and cells of muscle tissue.

Additionally, or alternatively, the system according to the invention may comprise a sensor for bioelectrical impedance analysis. Here, the determination of the body composition of the user can be measured. Here, one or more of the following, in particular, may be measured as measurement values: total body water (TBW), fat-free mass (FFM), lean body mass (LBM), fat mass (FM), body cell mass (BCM) and extracellular mass (ECM). This advantageously allows using such a whole body measurement of the user and triggering a stimulation pulse, in particular an EMS pulse, which has been modified in one or more stimulation pulse parameters, depending on the measurement value or control signal. The general composition of the tissue of the user lying under the sensor can be ascertained very well using bioelectric impedance analysis. This firstly renders it possible to ascertain the general state of the user, e.g. a fat/muscle ratio or the like, and, secondly, renders it possible to track during movements whether a muscle migrates to different positions or whether the composition of the tissue changes as a result of the movement. By way of example, subcutaneous fatty tissue will be pushed to the side by tensing and movement and the electrode will lie almost directly on the muscle. As a result, the stimulation can be appropriately increased or reduced during the contraction.

Additionally, or alternatively, the system according to the invention may comprise a sensor for determining the magnetoresistance. In the process, the determination of the magnetoresistance of the user can be measured. This advantageously allows using such a magnetoresistance of the user, and triggering a stimulation pulse, in particular an EMS pulse, which has been modified in one or more stimulation pulse parameters, depending on the measurement value or control signal. As a result of this, there may advantageously be, in particular, increased intensity and muscle power diagnostics by way of the magnetic field between the limbs.

Additionally, or alternatively, the system according to the invention may comprise a sweat sensor. Here, such as sweat sensor advantageously allows deductions to be drawn about the amount of liquid to be drunk during and/or after a stimulation application, for example a training session.

Additionally, or alternatively, the system according to the invention may comprise a GPS sensor for determining the GPS position of the user. This advantageously allows using a GPS position of the user, in particular an overall movement, the location of the user, the determination of the speed of the user and/or the height profile, in particular of a climb or assent, and triggering a stimulation pulse, in particular an EMS pulse, which has been modified in one or more stimulation pulse parameters, depending on the measurement value or control signal.

Additionally, or alternatively, the system according to the invention may comprise an acceleration sensor. This advantageously allows using detected movements of the limbs of the user for tracking techniques and/or coordination or general movements of the user, and triggering a stimulation pulse, in particular an EMS pulse, which has been modified in one or more stimulation pulse parameters, depending on the measurement value or control signal. As a result of this, there may advantageously be, in particular, an intensity increase and muscle power diagnostics by way of a magnetic field between limbs. By way of example, such an acceleration sensor may be arranged, in addition or as an alternative to a pressure sensor, in or on a shoe of the user. A measurement value of the acceleration sensor on the sole of the shoe can be measured and trigger a stimulation pulse, in particular an EMS pulse, which has been modified in one or more stimulation pulse parameters, depending on the measurement value or control signal. An increase in the measurement value on the sole of the shoe can trigger a stimulation pulse, in particular an EMS pulse, which has been modified in one or more stimulation pulse parameters, depending on the measurement value or control signal.

Additionally, or alternatively, the system according to the invention may comprise an electrocardiogram (ECG) sensor. This advantageously allows using measured cardiac values, in particular the heartbeat, comprising HRV, and hence analyzing defective functions or irregularities in the user, and triggering a stimulation pulse, in particular an EMS pulse, which has been modified in one or more stimulation pulse parameters, depending on the measurement value or control signal. The heart rate and, in particular, the HRV can be used, for example, as a stress marker in order to tone down the overall intensity of the training by way of the stimulation parameters and therefore serves, for example, as an “emergency stop” to avoid overexertion. On the other hand, it is possible to increase the overall load by adapting the stimulation parameters in the case of a load that is too low and a corresponding contradictory target. In an alternative preferred embodiment, the method comprises a measurement that is independent of cardiac parameters. In particular, the measurement values are heart-independent measurement values in an alternative preferred embodiment, in particular not cardiac values selected from heart rate, heartbeat, pulse and HRV.

Additionally, or alternatively, the system according to the invention may comprise a respiration sensor, in particular a strain gauge. This advantageously allows using the measured respiratory rate and/or depth of respiration, and hence also the analysis, documentation and power diagnostics, and triggering a stimulation pulse, in particular an EMS pulse, which has been modified in one or more stimulation pulse parameters, depending on the measurement value or control signal. In addition to possible power diagnostics and a possible estimation of the training intensity (in the case of a load that is too low and a corresponding contradictory target of the training, the overall load can be increased by adapting the stimulation parameters), it is also possible to temporally match the pulse such that the stimulation, as far as possible, is only used during the exhalation phases in order not to restrict the respiration, for example by way of a simultaneous stimulation of the accessory respiratory muscles, for example the chest.

Additionally, or alternatively, the system according to the invention may comprise a spirometry sensor, in particular an O₂ sensor and/or CO₂ sensor. To this end, the system according to the invention may comprise, in particular, a respiratory mask. Such a spirometry sensor advantageously allows the measurement of the metabolism. This advantageously allows using measured measurement values for setting training ranges, monitoring the load and the like, and triggering a stimulation pulse, in particular an EMS pulse, which has been modified in one or more stimulation pulse parameters, depending on the measurement value or control signal. In the case of a load that is too low and a corresponding contradictory target of the training, the overall load can consequently be increased by adapting the stimulation parameters. Consequently, it is possible to adapt the training load in an ideal manner in accordance with carried out power diagnostics, particularly within the scope of endurance.

Additionally or alternatively, the system according to the invention may comprise a lactate sensor. This advantageously allows using measured blood lactate values, and hence a more accurate training control as well, and triggering a stimulation pulse, in particular an EMS pulse, which has been modified in one or more stimulation pulse parameters, depending on the measurement value or control signal. In the case of a load that is too low and a corresponding contradictory target of the training, the overall load can be e.g. increased by adapting the stimulation parameters. Consequently, it is possible to adapt the training load in an ideal manner in accordance with carried out power diagnostics, particularly within the scope of endurance.

Additionally or alternatively, the system according to the invention may comprise a temperature sensor, in particular a thermometer. This advantageously allows using a measured body core temperature and an external temperature, and hence also calculating the calorie consumption, and triggering a stimulation pulse, in particular an EMS pulse, which has been modified in one or more stimulation pulse parameters, depending on the measurement value or control signal. By way of example, a temperature of the user, captured as a measurement value, may trigger an “emergency stop” function in order to avoid an overexertion of the body. Moreover, it is possible to make statements about the effects of the training that has been carried out.

Additionally or alternatively, the system according to the invention may comprise a sensor for determining blood sugar values and/or pH values of the user. This advantageously allows using measured blood sugar values and/or pH values, and hence also analyzing and documenting the physiological effects of the stimulation application and/or of the training, and triggering a stimulation pulse, in particular an EMS pulse, which has been modified in one or more stimulation pulse parameters, depending on the measurement value or control signal.

Additionally or alternatively, the system according to the invention may comprise an acoustic sensor. This advantageously allows using measured noises of the user, for example respiratory noises, in particular snoring noises, and hence also triggering a stimulation pulse, in particular an EMS pulse, which has been modified in one or more stimulation pulse parameters, depending on the measurement value or control signal. Such an acoustic sensor can be used for using the respiratory noises like the use of the strain gauge for measuring the respiration. Additionally or alternatively, such an acoustic sensor can be used for evaluating acoustic commands. By way of example, a spoken word, e.g. “stop”, may lead to a break in the stimulation pulses. In the case of a load that is too low and a corresponding contradictory target of the training, the overall load can consequently be increased by adapting the stimulation parameters. Consequently, it is possible to adapt the training load in an ideal manner in accordance with carried out power diagnostics, particularly within the scope of endurance.

Additionally or alternatively, the system according to the invention may comprise a time sensor, in particular a timepiece. This advantageously allows triggering a stimulation pulse, in particular an EMS pulse, which has been modified in one or more stimulation pulse parameters, depending on the measurement value or control signal. By way of example, the start or the modification of stimulation pulses or stimulation pulse parameters may be triggered at or after a predetermined period of time and/or depending on measurement values detected by further sensors.

Additionally or alternatively, the system according to the invention may comprise a contact sensor. In particular, such a contact sensor may comprise a capacitive element. This advantageously allows using measured contact, and triggering a stimulation pulse, in particular an EMS pulse, which has been modified in one or more stimulation pulse parameters, depending on the measurement value or control signal. By way of example, a stimulation pulse, in particular an EMS pulse, which has been modified in one or more stimulation pulse parameters, depending on the measurement value or control signal, may be triggered by contact, in particular a stroking movement on a capacitive element, or merely by tapping. This advantageously allows modification of stimulation pulse parameters, for example the stimulation pulse intensity, or else the selection of stimulation programs by the user.

Within the scope of the present invention, it should be understood that, advantageously, one or more sensors of the same type or of different types may be arranged in a system according to the invention.

In a preferred embodiment of the system or method according to the invention, the system comprises a plurality of sensors of different types. This advantageously allows the measurement of different measurement value types, in particular different physical or chemical variables. In particular, this allows measuring, monitoring, and documenting different vital data of the body of the user and, in particular, triggering different stimulation pulses which have been modified in one or more stimulation pulse parameters, depending on the control signal generated by the data processing unit.

In this context, it will be immediately understood by a person skilled in the art that, preferably, comparing a measurement value to a threshold may also comprise comparing a plurality of measurement values among themselves and/or with one or more thresholds.

A “threshold”, as used herein, is preferably understood to be a value which is used as a limit for processing a measurement value. By way of example, this can bring about a conversion of the measurement value into binary values. If a predeterminable relationship between measurement value and threshold is undershot, the measurement value is, for example, mapped to an output value of zero and, if the relationship is overshot, said measurement value is mapped to a constant output value, e.g. 1. By way of example, a pulse is triggered if the predeterminable relationship between measurement value and threshold is overshot.

It also lies within the scope of the invention to carry out a gradation of the stimulation parameters in accordance with the height of the measurement value. By way of example, a plurality of thresholds may be set in a system and/or method according to the invention, said thresholds having different stimulation pulses as a consequence. As a result of this, the pulse can be adapted even better to the respective situation. A data processing unit preferably comprises a comparator circuit. Here, a control signal is generated by the data processing unit depending on the comparison of the measurement value with a threshold or depending on the binary value resulting from the comparison of the measurement value with the threshold.

In a preferred embodiment of the present invention, the measurement value is measured at the user. In this context, it should be understood that measurement values are preferably measured directly at the user, for example by means of electrodes that have been placed on the user or which are in contact with the user. This advantageously allows a relatively compact configuration, in particular a portable/wearable configuration, of the system or method according to the invention and this does not restrict the user in terms of their freedom of movement.

In a preferred embodiment of the system and/or method according to the invention, the stimulation pulse parameters are selected from pulse type, intensity, duration of the stimulation pulse, frequency, ramp, pulse pause, individual pulse width, and individual pulse duration, rise time, and fall time. By way of example, a frequency may be approximately 2 to approximately 2500 Hz. By way of example, a duration may be approximately 2 to approximately 10 seconds or it may be a continuous pulse. By way of example, a pause may be approximately 1 to 5 seconds. By way of example, a ramp at the start may be approximately 0 to 0.3 seconds. By way of example, a ramp at the end may be approximately 0 to approximately 0.2 seconds. By way of example, an intensity may be approximately 25 to approximately 100 volt (peak-to-peak). By way of example, an individual pulse width may be approximately 100 to approximately 200 μs.

In a system or method according to the present invention, the pulse unit is suitable for triggering stimulation pulses and configured to modify one or more stimulation pulse parameters depending on the control signal.

The term “stimulation pulse”, as used herein, should preferably denote a pulse unit made of a plurality of individual pulses, which are triggered in quick succession with the same intensity or with different intensities. The term “individual pulse”, as used herein, preferably denotes a single process, the current values of which deviating significantly from zero only within a restricted period of time.

Such stimulation pulses that have been modified in terms of one or more of their stimulation pulse parameters may, in particular, be understood and characterized by means of the representation of voltage curves of the stimulation pulses, in particular those which are applied to the electrodes. In particular, the stimulation pulses may be represented by a plurality of rectangular curves of the intensities, in particular voltages.

Preferably, one or more individual pulses occurring in a stimulation pulse may have the same pulse type or different pulse types. Here, in particular, a pulse type is selected from bipolar pulse type and unipolar pulse type. Here, a bipolar pulse type should be understood to mean a changing pulse, i.e. an individual pulse with an intensity with a changing sign, in particular a sinusoidal or rectangular pulse.

Here, the “intensity” of a stimulation pulse denotes the maximum deflection of a stimulation pulse. Expressed differently, the intensity of a stimulation pulse is determined by the maximum deflection of the individual pulse or the individual pulses with the greatest deflection within a stimulation pulse.

In particular, this intensity of a stimulation pulse can be achieved directly at the start of the stimulation pulse, or after a sequence of ramp pulses that increase in terms of their maximum deflection. Likewise, at the end of the stimulation pulse, a stimulation pulse can be terminated immediately by the absence of pulses or said end can be obtained after a sequence of ramp pulses that decrease in terms of their maximum deflection. As used herein, a “ramp” should be understood to mean the characteristic gradient which is obtained from the maximum deflections of a sequence of such ramp pulses that in terms of their deflection increase at the start of the stimulation pulse or decrease at the end of the stimulation pulse. Here, the ramp is dependent on the duration and the maximum deflection of the ramp pulses before the start of the stimulation pulse until the intensity of the stimulation pulse is achieved, or dependent on the duration and the maximum deflection of the ramp pulses from the last individual pulse with the intensity of the stimulation pulse until the end of the stimulation pulse. The term “impulse pause”, as used herein, denotes the time duration between two successive stimulation pulses. The term “frequency”, as used herein, should be understood to mean how quickly periodically repeated individual pulses (with the maximum intensity) of the stimulation pulse follow one another. The term “pulse width”, as used herein, denotes the period of time of an individual pulse. Preferably, the pulse width is determined as the distance between beginning and end of an individual pulse. Preferably, a pulse width is approximately 25 to approximately 200 μs in the case of a stimulation pulse according to the present invention. Additionally, “rise time” and “fall time” may be important for the temporal sequence of addressing the muscle. The term “pulse duration” should be understood to mean the period of time for a stimulation pulse that is effectuated in the entirety thereof.

In an embodiment of the present invention, a plurality of stimulation pulses may be carried out overlaid on one another. By way of example, a first stimulation pulse may be defined by the following parameters: frequency: 50 Hz, duration: 5 seconds, pause: 3 seconds, pulse type: bipolar, ramp at start: 0.3 seconds, ramp at end: 0.2 seconds, intensity: 50 volts, individual pulse width: 100 μs and a second stimulation pulse may additionally be applied, the second stimulation pulse having the following parameters: frequency: 1000 Hz, duration: constant, pause: 0 seconds, pulse type: bipolar, ramp at start: 0 seconds, ramp at end: 0 seconds, intensity: 10 volts, individual pulse width: 50 μs. By way of example, this stimulation pulse is only triggered if the first stimulation pulse does not lead to stimulation at the same time. In this way, it is possible to overlay n stimulation pulses. Preferably, 1, 2 or 3 stimulation pulses are overlaid on one another. Moreover, the stimulation pulses can be applied as waves. Here, the intensity, but also frequency and individual pulse width, vary according to a predeterminable pattern. This pattern may repeat during the entire application.

In an embodiment of the present invention, one or more stimulation pulses may be configured as a continuous pulse. This should be understood to mean that quickly periodically repeating individual pulses at maximum intensity of the stimulation pulse are output in a continuous sequence during such a continuous pulse. In particular, quickly periodically repeating individual pulses at a maximum intensity of the stimulation pulse may repeat in a continuous sequence and with a pulse pause of 0.

In a further preferred embodiment of the method and/or system according to the invention, the stimulation pulse is an electronic stimulation pulse, in particular an EMS pulse. However, additionally or alternatively, it is possible within the scope of the present invention for a stimulation pulse to be provided as a mechanical pulse, in particular as a haptic pulse, in particular as a tactile pulse. The pulse may consist of a vibration. Preferably, the stimulation is an electrostimulation, in particular an EMS stimulation. An electrostimulation, as used herein, should preferably denote the stimulation of the body of the user by electrical stimulation pulses, in particular in the form of electric fields. In such an EMS pulse or such an EMS stimulation, the biological tissue of the user, in particular muscle cells, is excited directly by electrical stimulation pulses. These stimulation pulses are preferably selected to be significantly larger and longer than when stimulating nerves. In both cases, the functional electrostimulation can be carried out via the skin of the user by means of the pulse unit, in particular by means of electrodes connected to the pulse unit, preferably surface electrodes.

It is possible to strain different regions of the muscle fiber spectrum to different extents by changing the stimulation pulse parameters, in particular the pulse type, intensity, duration of the stimulation pulse, frequency, ramp, pulse pause, individual pulse width, and/or individual pulse duration, rise time, and fall time. In the case of frequencies of the individual pulses within the stimulation pulse of between 50 and 200 Hz, the fast muscle fibers are activated more while frequencies between 5 and 10 Hz tend to be suitable for improving the endurance capability, for which the slow muscle fibers are responsible. This form of stimulation is also referred to as EMS training herein.

Since the stimulation is not brought about by the physiological path, i.e. nerve system to muscles, but carried out in a direct way, the application of EMS, as known from the prior art, can only be used with restricted expediency; since it is usually carried out in combination with rest or only with simple movements, the coordination capability is not correspondingly improved. This disadvantage can be overcome by the present invention, in particular by a system according to the present invention that is configured as a portable/wearable system, and the method and system according to the invention also can be applied with free movement and, in particular, in conjunction with advantageously configured visualization units, and hence the coordination capability can also be appropriately improved.

The direct muscle stimulation with low-frequency stimulation current may be perceived as painful by a user and may be accepted to a lesser extent, in particular by persons who still have innervation. It therefore lies within the scope of the present invention to use medium-frequency current, in particular with frequencies above approximately 1000 Hz for a direct muscle stimulation. A person skilled in the art will immediately recognize that the sensitive load can be significantly reduced thereby since the electric resistance of the skin in relation to an electrical stimulus has an inversely proportional frequency dependence. Expressed differently, if the skin has a resistance of approximately 3000 ohm at 50 Hz, said resistance is only still approximately 30 ohm at 5000 Hz. Therefore, medium-frequency current forms are preferred for the system and method according to the invention, in which the stimulation pulse is an electronic stimulation pulse, in particular an EMS pulse. Therefore, a system or method according to the present invention will preferably provide stimulation pulses with frequencies of approximately 2000 Hz and/or modulated currents. Modulated currents may be provided, in particular, in the form of so-called modulated medium frequency (MMF).

In a further preferred embodiment of the system or method according to the invention, the system comprises a user interface comprising a visualization unit and/or at least one input means. Such a user interface allows the user of the system or method according to the invention, or a third party, e.g. a trainer or medical practitioner, to make adjustments on the system according to the invention or to gather information items. In particular, a method according to the invention can be set, issued or modified by means of such a user interface. In particular, a user interface can be configured in such a way that a program stored in the data processing unit, in particular comprising a method for controlling pulses during a stimulation on a user according to the present invention, can be selected or modified. This allows the system according to the invention and the method according to the invention to be adapted to the individual needs of the user.

Preferably, such a user interface comprises at least one input means. Such an input means is preferably suitable for the entry of values by a user and/or for selecting or changing a program stored in the data processing unit, said program in particular comprising a method for controlling pulses during a stimulation on a user according to the present invention. In particular, such input means can be provided by adjustment buttons, adjustment wheels, joysticks, sensor buttons or sensor fields, touchscreens or the like. In particular, a camera that is preferably configured to communicate with the system, in particular the data processing unit, may be provided as an input means. This renders it possible, for example, for a webcam to record the user during the stimulation. Also, such an input means can be configured as a sensor or part of a sensor according to the present invention, in particular a touch sensor. As an integral constituent part, the input means can be connected to the system, in particular to the electrodes, energy source, sensor, data processing unit, pulse unit or visualization unit. In particular, as an integral constituent part, such an input means can be connected to a textile that is comprised in the system, in particular an apparel piece. Alternatively, the input means can also be configured as a separate component. Preferably, the input means may be configured as a remote control in this case. As a result, it is not necessary, for example, for the person controlling the system and/or the method to be situated in the same room as the user. Alternatively or additionally, the system can be controlled by means of remote data transmission.

A communication between the input means and the system can be obtained by means of a wired connection with a mechanical effect, e.g. Bowden cable, air break switch, tethered flight or the like; by means of a wired connection with an electrical effect; or by means of wireless input means, for example by means of sound, ultrasound, radio waves, infrared radiation (IR), the Internet, a LAN, an intranet, a WLAN or the like. In particular, such a system may comprise interfaces which facilitate the communication of the input means with the system, in particular with the data processing unit.

Also, such an adjustment means can be configured as part of a visualization unit. Preferably, such a user interface comprises a visualization unit in addition or as an alternative to the at least one input means. In particular, such a visualization unit can be configured and connected to the system, in particular the data processing unit, in such a way that feedback is presented by means of the visualization unit depending on the stimulation being carried out at a user, in particular an EMS application, and/or depending on the measured values and/or depending on the relationships of the measured values to the threshold.

The term “visualization unit”, as used herein, should preferably denote an elucidation device that brings abstract data of the system and/or method according to the invention, in particular comprising control pulse parameters, control pulse programs, measurement values, thresholds and the like, and relationships into a form that can be grasped by the user, preferably that can be grasped graphically or visually. In particular, a visualization unit may facilitate the reproduction of the abstract data of a system and/or method according to the invention in a visual and/or acoustic and/or haptic form that can be grasped by the user. To this end, such a visualization unit may, in particular, comprise a screen, a touchscreen, vibration elements and/or loudspeakers.

In an embodiment of the method and/or system according to the invention, as an integral constituent part, a visualization unit can be connected to the system, in particular to the electrodes, energy source, sensor, data processing unit, pulse unit or input means. In particular, as an integral constituent part, such a visualization unit can be connected to a textile that is comprised in the system, in particular an apparel piece. Alternatively, the visualization unit can also be configured as a separate component. Preferably, the visualization unit may be configured as a wireless visualization unit. By way of example, this can facilitate a visualization of the abstract data of the system separately from the user. By way of example, the user can track the visualization of the abstract data on a television set or on a screen, or on a wristwatch, without being securely connected thereto, for example by means of cables or wires. Alternatively, a third party can monitor the data on such a visualization unit independently of and, in particular, spatially separate from the user. As a result, it is not necessary for the person who is monitoring and optionally controlling the system and/or method to be in the same room as the user. In particular, the values that are measured by means of the sensors, in particular the vital values of the user, may be transmitted in real time or with a delay. By way of example, the values can be transmitted to a trainer or a medical supervisor during an application. This allows capturing and storing of the measurement values and, in particular, monitoring of the application. From this, it is possible to draw conclusions about the application carried out by the user, said conclusions, in turn, allowing the application behavior of the user to be optimized. By way of example, athletic movements, for example within the scope of golf, basketball or the like, can be analyzed by the user themselves or by third parties and can be improved. In addition, the system according to the invention allows the collected data to be stored and/or to be transmitted to a storage medium, in particular a cloud as well, by means of a suitable data link and storing said data there.

A communication between the visualization unit and the system can be obtained by means of a wired connection with an electrical effect, or as a wireless input means, for example by means of sound, ultrasound, radio waves, infrared radiation (IR), the Internet, a LAN, an intranet, a WLAN or the like. In particular, such a system may comprise interfaces which facilitate the communication between the visualization unit and the system, in particular the data processing unit. In a preferred embodiment, the visualization unit comprises a monitor, a screen, a touchscreen or a projector. This advantageously allows a relatively complicated and/or large-area representation. In an alternative preferred embodiment, the visualization unit comprises a timepiece, in particular a watch, with a display. This particularly advantageously allows a timepiece to be worn, in particular during sports, and allows a relatively compact presentation. In a further alternative preferred embodiment, the visualization unit comprises spectacles, in particular 3D goggles. This advantageously allows the presentation directly in front of the eye of the user. Here, the presentation of a virtual space, in particular in a 3D view, can particularly advantageously also be facilitated. In a further alternative, the visualization unit can comprise LEDs or OLEDs.

In a preferred embodiment, the system according to the invention comprises a visualization unit for presenting a virtual reality, in particular in a 3D view. In an embodiment, the method according to the invention comprises a step of providing a virtual space, in particular in a 3D view, and, optionally, a step of visualizing the user and/or virtual surroundings of the user in a virtual space, in particular in a 3D view. In a preferred embodiment, the system according to the invention comprises a data processing unit that is adapted to present a virtual reality on a visualization unit. Here, the presentation of a virtual reality, in particular in a 3D view, preferably comprises the presentation and simultaneous perception of reality or fictitious surroundings, and the physical properties thereof in interactive virtual surroundings that are generated by computer in real time. Expressed differently, in the case of e.g. a user who guides the hand to the shoulder by bending the elbow, as in the case of a biceps curl movement, a sensor, e.g. a movement sensor, can recognize and measure this movement. The data processing unit can compare the measurement value to a threshold and generate a control signal for the pulse unit if the measurement value and the threshold lie in a predeterminable relationship to one another. Accordingly, the pulse unit can trigger stimulation pulses which are changed in terms of one or more stimulation parameters, depending on the control signal. By way of example, the data processing unit can generate a control signal for the pulse unit and increase the pulse intensity with increasing shortening of the angle between forearm and upper arm. At the same time, a virtual reality is provided for the user, in which the user sees themselves, for example on data goggles, in respect of how they carry out the movement with a dumbbell, even though they are not, in fact, holding a dumbbell in their hand. As soon as the user has straightened the arm again, the pulse unit can terminate the triggering of stimulation pulses.

In this context, it should be understood that the method and/or system according to the invention can be particularly advantageously configured by virtue of, firstly, abstract data of the system and/or method according to the invention, in particular comprising control pulse parameters, control pulse programs, measurement values, thresholds or the like, and relationships being able to be presented in a form that can be gathered, preferably gathered graphically or visually, by the user and, secondly, by virtue of, additionally or alternatively, it being possible to present to the user themselves, a real or fictitious training partner and/or a virtual space surrounding said user during the stimulation. This advantageously allows the user to be put into an artificial reality during a stimulation application. This can increase the acceptance and the well-being of the user in a medical application or in sports-related training, for example by virtue of the user being presented with more pleasant surroundings. Secondly, the system according to the invention can lead to more effective training by way of such a representation by virtue of a real training partner and/or a fictitious training partner being presented to the user themselves. Finally, the system and/or method according to the invention can also be configured to carry out games, in particular video or computer games.

In particular, an automatic trainer who provides the user with instructions depending on the measurement values, in particular on the measurement values during the movement, detected by means of the sensor and who hence optimizes the application behavior, in particular the training behavior, of the user may be provided by means of the method and/or system according to the invention and, in particular, by means of virtual reality.

In a preferred embodiment of the invention, the user themselves is presented in a virtual reality by means of the visualization unit while said user is carrying out training. The system used by the user in the process preferably comprises sensors which facilitate representing the movement of the user by means of the visualization unit. Then, the parameters of the stimulation pulses can be adapted depending on the movement of the user. Then, the sensor preferably is a movement sensor. The pulse is preferably an EMS pulse. Hence, the user is preferably able to control their avatar, i.e. their representation in the visualization unit. Depending on the movement of the user, the system may trigger pulses to the user.

In particular, provision is made according to the invention for the apparatus to be configured in such a way, or for the method to comprise, that a user who uses the apparatus, in particular wears an apparel piece, controls the stimulation and/or feedback (modified pulse parameters) by way of their movement. This movement and/or the feedback are preferably visualized by the apparatus.

The user interface, in particular the visualization unit and/or the input means, may be fastened to a band which can be worn on the hand in one embodiment of the system according to the invention. Here, it is preferable for the band to be configured in such a way that it extends over the palm of the hand and the back of the hand and that it is configured with a thumb loop. The thumb loop affixes the band. As a result, it is possible to place the user interface in an easily visible place even if a textile that is comprised by the system, in particular an apparel piece, has comparatively long sleeves—as is common in sportswear. The band can be provided with a hook-and-loop fastener for individual adaptation purposes.

In a further preferred embodiment of the system or method according to the invention the system comprises an energy source. In particular, an energy source that is configured to recuperate energy by movement is provided in one embodiment of the system and/or method according to the invention. Additionally or alternatively, crystals that are integrated into a textile, in particular an apparel piece, that is comprised by the system may serve to produce energy. Additionally or alternatively, thermal energy, in particular thermal energy produced by the user during the application of the system and/or when carrying out the method according to the invention, for example as a result of an increase in the body temperature, may serve as an energy source. Additionally or alternatively, the system may contain an energy source which is configured to convert solar energy into electrical energy; by way of example, the system can use solar energy by appropriate panels on the surface of a textile, in particular of an apparel piece, in order to provide useful energy to the system and/or individual components. Also, the employed yarn may be suitable for converting solar energy into electrical energy.

Such an energy source allows the system according to the invention to provide sufficient useful energy for the use according to the invention. In particular, the sensor, data processing unit and pulse unit, and further components of the system may comprise a common energy source or different energy sources.

In the case of embodiments of the system and/or method according to the invention in which the stimulation pulse is an electrical pulse in particular, it may be advantageous to provide the pulse unit, in particular electrodes (to the extent that these are present), with a separate energy source that is assigned to the pulse unit. In particular, dedicated energy sources in the form of energy storage means, in particular accumulators, may be assigned in each case to the pulse unit and the optionally present individual electrodes or electrode pairs.

In a further preferred embodiment, the one or more energy sources may obtain energy without wires, for example by movement or induction.

In a further preferred embodiment of the system or method according to the invention, the system is a portable/wearable system.

Preferably, a system according to the present invention is a portable/wearable system. The term “portable/wearable system”, as used herein, should preferably denote a system, the components of which, in particular the sensors, data processing unit, pulse unit, conductor, electrodes of which, to the extent that these are present, can be worn or carried by the user. This advantageously allows a relatively compact, in particular portable/wearable configuration of the system or method according to the invention and does not restrict the freedom of movement of the user. In particular, the whole system or individual components of the system, in particular the data processing unit, pulse unit, electrodes that, in particular, are connected to the pulse unit, the user interface, the information unit, the visualization unit, may be configured as portable/wearable components.

In a further preferred embodiment of the system or method according to the invention, the energy source is a portable energy source. In particular, accumulators may be provided as a portable energy source in order to provide useful energy to the system or individual components of the system, in particular the pulse unit, in particular electrodes connected to the pulse unit.

In a further preferred embodiment of the system or method according to the invention, the electrodes, the energy source, the sensor, the data processing unit and/or the pulse unit are connected to a textile, in particular an apparel piece.

This advantageously provides a system in which the user may carry or wear the system or individual components thereof with the textile, in particular an apparel piece, without the user being restricted in terms of location and/or their freedom of movement.

Here, the scope of the present invention includes the system, in particular the electrodes, the energy source, the sensor, the data processing unit and/or the pulse unit, being securely connected to the textile. This can be obtained by means of a suitable connection means, for example by means of a stitched connection, by means of a magnetic connection, by knitting-in or the like. Alternatively, the scope of the present invention also includes the system, in particular the electrodes, the energy source, the sensor, the data processing unit and/or the pulse unit being detachably connected to the textile. This can be achieved by means of suitable connecting means, for example by means of hook-and-loop fasteners, belts, buckles, snap fasteners, magnets or the like.

By way of example, the energy source is provided as an elastic, thread-like battery in an embodiment of the present invention. Here, the battery may be worked into the fabric of the textile and hence be wearable.

Likewise, e.g. the pulse unit, in particular electrodes connected to the pulse unit, may be worked into the fabric of the textile and hence be wearable.

Alternatively or additionally, the individual components of the system may have a flexible configuration. Alternatively or additionally, the individual components have a watertight embodiment such that the entire system can be washed by hand or in a washing machine.

A particularly preferred embodiment relates to a textile that is configured as a so-called wing, i.e. an apparel piece which has two oversleeves that are interconnected via the back. Each oversleeve of the wing according to the invention preferably has a pulse unit in this case and, in particular, electrodes that are connected to the pulse unit. In particular, such a pulse unit may comprise at least two electrodes. The pulse unit, in particular the electrodes, may be arranged e.g. at the upper arms, biceps/triceps or on the back.

An alternative embodiment relates to a textile that is configured as a cuff for the extremities or the trunk. In particular, such a cuff can have at least two zones: a conductive zone and, placed thereover, a nonconductive zone. By way of example, the electrodes can be embodied as ring electrodes and/or as electrode surfaces. Such electrodes can be advantageously placed onto the muscles. A further particular embodiment relates to specific electrodes for the tongue or the oral cavity.

A further embodiment provides for the distribution of an electrode pair to two persons. In this case, stimulation is present only when these two persons are in contact. In particular, the electrodes can be arranged in such a way that a stimulation pulse will be primarily perceived at the point of contact.

In a special embodiment, a textile is an apparel piece destined for animals, in particular for camels, dogs or horses, and adapted to the anatomical peculiarities of the animal.

In an embodiment of the system or method according to the invention, in which the electrodes, the energy source, the sensor, the data processing unit, and/or the pulse unit are connected to a textile, in particular an apparel piece, such a textile can be adapted or destined for the upper body or lower body of the user; consequently, this may be, for example, a T-shirt, a long-sleeved shirt, a hat, a cheek and/or face mask, a tank top, briefs, a brassiere, a sole, a pair of stockings, a pair of shoes or a pair of trousers. In a further preferred embodiment of the system or method according to the invention, the system, in particular the pulse unit, comprises at least two electrodes.

In particular, the system according to the invention for controlling stimulation pulses during a stimulation on a user may comprise at least one data processing unit, which is configured to generate a control signal for a pulse unit, and a pulse unit, wherein the pulse unit is suitable for triggering stimulation pulses and wherein the pulse unit comprises at least one channel, wherein at least two electrodes are connectable to the channel and are controllable independently of one another.

In this context, it should be understood that such a system according to the invention may comprise, in particular, a textile or a textile that is configured as an apparel piece, preferably a pulse unit which, in particular, may comprise an EMS device having one or more channels. Two or more electrodes can be connected to these channels. The assignment is flexible. Alternate electrodes may be assigned to a channel by way of a further MC or router. In its simplest case, different electrode pairs are addressed in succession, wherein the respective pairs remain the same (monogamous solution). In a further embodiment, the assignments of the electrodes can change (polygamous solution).

A system according to the invention, in particular an EMS system, can be simplified if a stimulation channel can be applied to alternate electrode pairs by means of a plurality of relays.

Advantageously, this permits a relatively cost-effective embodiment of the system. In particular, an individual channel may comprise a plurality of relays and this allows stimulation pulses to be guided to each connected electrode or to each connected electrode pair. Here, the number of relays depends on the number of muscle groups to be stimulated. By way of example, two relays are required to switch between two muscle groups to be stimulated. Switching between relays after a certain period of time allows all desired muscles of the body to be stimulated.

Preferably, the electronics of the system according to the invention are configured in such a way that up to 12 muscle groups can be trained. In order to be able to actuate the electrodes of the respective muscle groups independently of one another, it used to be necessary to provide up to 12 channels to this end in the electronics according to the prior art. However, this is comparatively expensive.

In order to be able to design the electronics or the controller of the system according to the invention in a relatively cost-effective manner for the user, a system according to the invention, in particular a textile or a textile that is configured as an apparel piece, may have only one channel and comprise a relay and a microcontroller, by means of which the individual electrodes are actuatable in succession. Alternatively or additionally, the electrodes may also have any assignment, for example first left abdomen—right abdomen, then left abdomen—right chest.

In a preferred embodiment of the system and/or method according to the invention, the system, in particular a textile or a textile that is configured as an apparel piece, comprises at least one or more channels for actuating the electrodes. Here, each channel may be controlled independently of the other channels in view of the stimulation parameters, in particular pulse type, intensity, duration of the stimulation pulse, frequency, ramp, pulse pause, individual pulse width, and/or individual pulse duration, the rise time and fall time, and polarity. Only providing this before the start of a stimulation application or during a stimulation application also lies within the scope of the present invention. Moreover, it lies within the scope of the present invention to provide a curve of these parameters on the basis of defined values.

Additionally or alternatively, provision can be made of a channel which is provided with appropriate relays and microcontrollers and thereby forms electrode groups which are separately actuatable at the same time, wherein electrodes of a group may likewise be actuated in succession. These can be combined as desired; that is to say, there may be more than one group.

In particular, a system according to the invention may allow at least one channel switch. A method according to the invention may comprise a step of switching channels between two or more electrodes or electrode pairs. Such a channel switch allows stimulation of the whole body of the user by means of only a few channel electronics units, preferably by means of a single channel electronics unit. A system in accordance with the present invention preferably comprises a one-channel system. Such a system is relatively cost-effective and relatively compact. Here, a person skilled in the art will acknowledge that the brain of the user, in particular of a human user, will not process signals in the millisecond and microsecond range. If switching between the individual electrodes is carried out quickly enough in such a one-channel system, for example each millisecond, then 10 channels can be worked by only one stimulation channel at a frequency of 100 Hz without this being noticed, and so the user is under the impression of a stimulation relating to the whole body. By way of example, such a switchover may be carried out between individual electrodes or electrode pairs, i.e., for example, from electrodes that are arranged on the chest of a user to electrodes that are e.g. arranged on the abdomen, or from electrodes that are arranged on the right chest side to electrodes that are arranged on the left chest side. Also, such a system may comprise electrodes that are arranged on the spinal column and that switch from the upper spinal column region to the lower spinal column region, or vice versa, in order e.g. to treat back pain. Therefore, such a one-channel system can advantageously replace a multi-channel system.

In addition, a channel switch may also be carried out in the case of more than one channel electronics unit. This should be understood to mean that at least one channel actuates at least two electrodes. A person skilled in the art will immediately understand that, advantageously, the number of electrodes and, like above, the number of groups can be increased. Here, there may be partly a fixed assignment and partly a flexible one.

Therefore, a channel switch can be used particularly advantageously in order to actuate the pulse unit, in particular various electrodes, with stimulation pulses. This allows triggering the stimulation pulses at pulse units, in particular electrodes, in very different regions of the body of the user and hence applying a stimulation pulse to any desired muscle.

In a further embodiment of the present invention, the components of the system according to the invention, in particular pulse unit, are not connected to the textile, in particular not to the apparel piece of the user; i.e., in particular, they are not part of the apparel piece. In such an embodiment, the apparel piece preferably has an apparatus which secures the components of the system according to the invention, in particular the pulse unit, against slipping. By way of example, this apparatus can consist in a pocket which has a detachable connecting means such as a hook and/or loop fastener. This apparatus can be used for all articles that should be carried along loose, even independently of a stimulation application. In an alternative embodiment, the components of the system according to the invention, in particular the pulse unit, may be connected by means of magnetic closing and/or latching to the apparel piece. In general, the components of the system according to the invention, in particular pulse unit, may be part of the apparel piece or merely be connected by cable to individual electrodes or all electrodes of the apparel piece.

Here, the electrodes can be securely connected to the textile, in particular the apparel piece, for example directly stitched or drop stitched onto the textile, or applied onto a carrier material in advance, said carrier material in turn being connected, in particular sewed, stitched, adhesively bonded, laser bonded or welded, to the textile.

In a preferred embodiment of the system and/or method according to the invention, the electrode comprises a conductive yarn or is formed from a conductive yarn. In particular, electrodes that are embodied to transfer an electronic stimulation pulse, in particular an EMS pulse, can comprise a conductive yarn or be formed from a conductive yarn. By way of example, the conductive yarn can be a metal-containing thread, in particular a metal-coated thread. The thread is preferably a polymer thread, in particular selected from polyamide, polyester or else polypropylene. The metal is selected from metals with electric conductivities of at least 1 and at most 80 S/m, more preferably at least 40 S/m. The metal is preferably copper, silver or gold. Alternatively, the already coated yarn may still be surrounded by a titanium layer. The latter is preferably a few atoms thick.

Textiles according to the invention, in particular apparel pieces, may comprise conductive zones with electrodes and zones without electrodes, in particular as part of the pulse unit. Here, zones with electrodes comprise electrodes and are therefore conductive. The zones without electrodes are preferably nonconductive. This means that these electrode-free, nonconductive zones have a conductivity of less than 0.5 S/m in the dry state.

The zones with electrodes can preferably contain up to approximately 75%, more preferably up to approximately 80%, 85%, 90%, 95%, more preferably up to 100% conductive yarn. In a preferred embodiment, the zones with electrodes have up to approximately 80% conductive yarn. Here, it should be understood that a percentage specification in relation to the component of conductive yarn relates to the component of the conductive yarn on the textile in the corresponding zone by weight. In order to ensure ideal conductivity, it is advantageous to provide a component of conductive yarn in the zones with electrodes which is at least 10%, more preferably at least 20% and very preferably at least 30%. The various zones can merge seamlessly into one another in order to increase the comfort of wear. In particular, they can be circular knitted.

In one configuration of this invention, the conductive yarn, in particular in the region of at least a zone with electrodes, is combined with a hydrophilic yarn. This preferably means that the conductive yarn is warp-knitted, weft-knitted, embroidered or folded together with the hydrophilic yarn. This measure was found to be particularly advantageous if the electrode requires moisture in order to overcome the skin resistance. The hydrophilic yarn can be selected from the group consisting of viscose, cotton, wool or else a hydrophilically designed synthetic yarn.

The ratio of hydrophilic yarn to conductive yarn is preferably at least 1:10, more preferably at least 1:4. If a component of hydrophilic yarn that is too low is used, the desired effect does not occur. If a component of hydrophilic yarn that is too high is used, the component of the conductive yarn is proportionately too low, and so the desired conductivity cannot be obtained. Therefore, the aforementioned ratio is preferably restricted to at most 1:1, more preferably at most 1:2. What is meant here is the mass ratio in the relevant zone.

In a preferred embodiment, the electrodes may be provided with a moisture-storing layer as an alternative or in addition to the hydrophilic yarn. The layer is preferably arranged between the skin and the electrode. That is to say, it is preferably on the inner side facing the body in the apparel piece according to the invention. In particular, the additional layer can be a layer that consists of a moisture-storing material, preferably in the form of a non-woven fabric.

It is preferable for the electrodes to be detachably connected to the textile according to the invention, in particular to the apparel piece. The electrodes may preferably be detachably fastened to the inner side of the apparel piece. To this end, the electrodes can be provided with hooks on their outer side. The apparel piece then preferably has loops at the corresponding position of its inner side, which establishes a detachable connection with the hooks. Corresponding embodiments which have loops on the electrode and hooks on the apparel piece are also part of the invention. The detachable connection offers the advantage that the electrodes can be attached to very different positions. As a result, the apparel piece becomes more flexible. Furthermore, defective electrodes can easily be replaced or the type of electrode can be adapted to the desired use; as a result, the apparel piece can be used longer and more flexibly. As an alternative or in addition to the hook-and-loop connection, use can be made of magnets or other detachable connections.

The electrodes can be manufactured from a conductive polymer. Silicone which is designed to be conductive by the addition of titanium particles is a preferred conductive polymer. Instead of a smooth surface, the surface of the electrode is preferably roughened in order to avoid slippage during use. In the case of the conductive polymer, this is preferably obtained by the use of a correspondingly rough matrix when forming the electrode. Alternatively, the conductive polymer can also be equipped with a rough surface after its production, in particular by mechanical post-processing. The electrode is preferably adapted to the body form of the user. What this means is that the electrode is preferably fitted to the anatomy of the body.

Particularly in the case of a conductive polymer, the electrodes that are used according to the invention may have a multilayer structure. In particular, multilayer means two layers, three layers or four layers. Here, the layer structure may comprise one or more conductive and/or one or more nonconductive layers.

In the case of a multilayer structure of the electrode, one layer can once again consist of a fabric layer. Here, the fabric is preferably selected in such a way that its stretch or elasticity approximately corresponds to that of the material of the electrode. This is particularly important in the case of electrodes which comprise a conductive polymer, as such polymer electrodes can tend to tear and the fabric layer can reduce the tear propagation. More preferably, the elasticity is 10% to 30%. The fabric layer in the electrode, which is preferred according to the invention, may also serve to affix a connection that can serve to introduce the stimulation current. Independently of an optional fabric layer, the electrodes according to the invention preferably have a connection for introducing the stimulation current. The fabric layer may be manufactured from a conductive material, in particular from the above-described conductive yarn. This additionally ensures that the current distributes uniformly or as desired within the electrode, which was found to be advantageous, particularly in the case of electrodes with conductive polymers. This applies in particular if the fabric layer is used for affixing a connection for introducing the stimulation current.

The configuration of the electrodes according to the invention allows these to be configured to be particularly thin. This applies, in particular, to electrodes which comprise a conducting yarn or conductive polymer. The electrodes according to the invention can preferably have thicknesses in the range from 0.1 mm to 3 mm. Additional contact pressure can be produced by an optional cushion between the outer side of an electrode and the inner side of the apparel piece. This is preferred for concave body regions in particular. Consequently, a preferred embodiment of this invention relates to an apparel piece which has at least one cushion between an outer side of an electrode and an inner side of the apparel piece, in particular in a concave body region such as between the breasts. The cushion can be an inflatable cushion, for example an air chamber, a balloon or the like. The cushion is arranged between the inner electrode and the outer textile layer of the apparel piece and preferably detachably connected to the apparel piece.

If this document refers to an “inner side”, then this means the side facing the body of the user if there is any doubt. If reference is made to the “outer side”, this thus means the side that faces away from the body of the user.

In preferred embodiments of the invention, the apparel piece comprises at least an outer ring electrode and at least an inner ring electrode, in particular two outer ring electrodes. In addition to the preferred use with bipolar currents, these electrodes are also suitable for the use with unipolar currents. The entry of the current into the body should preferably be brought about by way of the larger electrode. Stimulation units which only facilitate unipolar pulses allow a further reduction in the system complexity.

The apparel piece according to the invention preferably has different compression zones or a gradual compression.

In a further preferred embodiment of the system or method according to the invention, the system comprises conductors for electrically connecting the pulse unit and electrodes. Such conductors allow the transfer of a stimulation pulse that is produced by the pulse unit from the pulse unit to the electrodes. Such conductors may comprise a combination of a polymer, in particular silicone, and conductive twisted yarn, yarn, cord or the like. The conductive medium is preferably encased by silicone in a non-rigid or springy manner. Electrodes may be connected to the conductors in a non-detachable manner, for example by means of stitching, or in a detachable manner, for example by means of a snap fastener. The conductors themselves may also be connected to the textile, in particular the apparel piece, in a detachable or non-detachable manner. In one embodiment, in which the conductors are connected to the textile in a non-detachable manner, the conductors are embroidered onto the textile, in particular the apparel piece. In the process, the insulated or non-insulated conductors may be arranged on the textile, preferably in a meandering manner, in particular in a non-rigid or springy manner.

In a preferred embodiment of the method and/or system according to the invention, the method and/or system is suitable for learning a predeterminable sequence of movements. In an embodiment, the system, preferably the data processing unit, comprises a sequence of stimulation pulses that corresponds to a sequence of movements. In an embodiment, the method comprises a step of generating stimulation pulses in a sequence corresponding to a predeterminable sequence of movements. By way of example, the user's attention is drawn to a deviation from a predetermined sequence of movements by way of EMS pulses or the muscles are contracted by a suitable stimulation sequence in such a way that the user carries out the predetermined movement. Here, it is considered to be particularly advantageous within the scope of the present invention that the system and/or method according to the invention is suitable for treating paraplegics who, as a result of this, can learn or carry out sequences of movements again through their own efforts.

To this end, a sequence of movements of a user can be predetermined or predeterminable in the system, in particular in the data processing unit. Here, the method according to the invention advantageously allows describing a sequence of movements by the temporal sequence of the muscle contractions. To this end, a corresponding sequence of movements, which has been stored in the system, in particular in the data processing unit, may prompt or assist a user to carry out precisely this sequence of movements. By way of example, the round step when cycling, a golf swing, a pre-contraction when running, just before the user contacts the floor, or the like may be predeterminable as a sequence of movements in the system, in particular in the data processing unit. This advantageously allows learning previously unknown sequences of movements. In particular, such a sequence of movements can be adaptable to the body of the user, in particular the body contour, the weight or the height.

Furthermore, the scope of the invention contains measuring and/or documenting the correct execution of the sequence of movements by the user by means of sensors. As a result, the user can monitor the movement to be learned and their progress. In particular, the user can obtain appropriate feedback and/or be corrected by means of the system and/or method according to the invention if the sequence of movements is executed incorrectly. To this end, an ideal sequence of movements, in particular, may be stored in the system as a threshold. As a result of this, a comparison of the actual sequence of movements recorded by means of the sensor with a sequence of movements predetermined as threshold can generate a control signal if the actual sequence of movements of the user, measured as a measurement value, and the predetermined sequence of movements, as a threshold, have a predefined relationship to one another, for example if the user deviates too strongly from the predetermined sequence of movements. As a consequence, one or more control pulse parameters may be modified depending on the control signal in order thus to draw the user's attention to the incorrect sequence of movements and/or correct the latter.

In a preferred embodiment of the method and/or system according to the invention, the method and/or system is suitable for giving the user feedback about a game situation in a computer or video game. In a preferred embodiment of the system according to the invention, the system, in particular the data processing unit, comprises a games interface. By means of such a games interface, the system is connectable to a computer game or video game. By way of example, a stimulation pulse may be used in a computer game in order to elucidate a game situation, in particular hits, up to the intermittent partial immobilization of the player or players. By way of example, the user can play the video or computer game and specific game situations may trigger one or more stimulation pulses, which simulate certain game situations, for example a hit. To this end, there may be a provision of further sensors which are suitable to transfer emotions to the game or further users involved in the game. This application can also be transferred to laser tag or similar games. In particular, such a video game or computer game can provide a virtual space within the scope of the system and/or method according to the invention.

A method for controlling stimulation pulses during a stimulation on a user according to the present invention using a system, in particular a portable/wearable system according to the present invention, wherein a pulse unit triggers one or more stimulation pulses, comprises the following steps:

a. measuring a measurement value,

b. comparing the measurement value to a threshold,

c. generating a control signal if the measurement value and the threshold have a predeterminable relationship to one another,

d. modifying a stimulation pulse parameter depending on the control signal.

In a preferred embodiment of the method according to the invention, steps a to d are repeated at least every 10 minutes during the duration of an application. In particular, step a of the measuring of a measurement value can be repeated regularly in a continuous or discontinuous manner. In a further preferred embodiment of the method according to the invention, a stimulation pulse is characterized by a frequency in the range of 1 to 100 Hz.

All embodiments of the method and/or system in accordance with the present invention exhibit the advantage that, for the purposes of controlling stimulation pulses, in particular EMS pulses, the mobility and handling of such a system and/or method are increased. By way of example, EMS may be combined with sports-specific training. Thrombosis prophylaxis, for example on long-haul flights, becomes possible. The invention is suitable for specific use for or during sports prior to (warm-up, activation), during (increase the effectiveness of the training) or after the sport (improvement of regeneration). Moreover, the method and/or system according to the invention allow, in particular, feedback to the user. On the basis of the measurement values ascertained by way of the sensors, the data processing unit calculates stimulation pulses taking into account thresholds that are defined in advance, said stimulation pulses being transferred to the body by way of the pulse unit, in particular by way of electrodes connected to the pulse unit, and activating the corresponding body regions. Particularly advantageously, the method and/or system according to the invention provides an option of allowing a mobile and simple use of the stimulation application. In particular, the system according to the invention is distinguished by a high quality of the materials, the improved option of documenting training achievements, improved training control, for example by way of a virtual online coach, sports-scientific backgrounds, and individual designs of the training and of the stimulation pulses.

Multichannel systems with one to 12 channels may address all muscle groups of the body. Here, each channel is isolated from the other channels and individually actuatable. The channels may be electrically isolated from one another.

Each channel can be rated up to 50 V-100 mA@500 ohm. This is the maximum power that a user can obtain from a channel in order still to satisfy the safety criteria. However, channels with up to 100 V may also be provided.

In stationary systems, in particular EMS systems, size plays a subordinate role. However, if all muscles should be addressed during a mobile application, for example during a run, size and weight of the system are an important factor. The system according to the invention overcomes these problems by virtue of being able to arrange a maximum number of channels in relatively little space. Hence, the system according to the invention allows just as many groups of muscle to be addressed as stationary multichannel apparatuses from the prior art, but also permits the mobile portable/wearable application.

Furthermore, the data processing unit may be embodied as a mobile application, in particular of a cellular telephone, computer or tablet PC, said mobile application allowing the method according to the invention, in particular a training application with the system according to the invention, to be selected, monitored and set. Here, use can be made, in particular, of Bluetooth, Internet, WLAN or other wireless communication methods, which facilitate real-time communication between the data processing unit and pulse unit. In particular, the system may, in the case of communication transfer problems, comprise a further data processing unit, in particular a further user interface, and, in particular, further input means which allow at least basic adjustments to be undertaken on the system. By way of example, in the case of an outage of the data processing unit configured as a cellular telephone, a system, for example a track suit, may have a further user interface in order to modify the intensity of stimulation pulses or in order to switch off the stimulation pulses.

Additionally or alternatively, current-limiting units may be integrated into the system in order to protect the user from unwanted damage to muscles or skin as a result of current pulses that are too strong. Furthermore, continuous monitoring of the stimulation pulses at the muscles may be provided by the system, said monitoring protecting the user from unwanted, suddenly occurring high current intensities.

The capability of the system according to the invention of also providing higher frequencies renders it moreover applicable for muscle regeneration, relaxation treatment and pain treatment. Conventional devices are only able to provide approximately 1 Hz to 150 Hz. The system according to the invention allows frequencies from approximately 1 Hz to 2000 Hz. Here, higher frequencies, such as e.g. 2000 Hz, preferably find use in special programs that are recommended by experts. However, provision is made, in principle, for a user to be able to regulate the programs, optionally predetermined in the data processing unit, in the range from approximately 1 Hz to approximately 150 Hz, and so the use of the system according to the invention remains safe for the average user.

The strength of elderly humans can be advantageously improved and maintained longer or be rebuilt using simpler means with the aid of the method and/or system according to the invention, in particular if use is made of EMS.

For these cases, the system and/or method according to the invention, particularly within the scope of EMS training, provide a very good option of restoring the lost quick muscle fibers, especially in the extremities. Moreover, general stabilization could be obtained in the trunk region which likewise has a positive effect on the overall bodily constitution. This lays the foundation for a fall-free future.

The visualization unit may be configured to produce an image of the person training, such as e.g. an avatar. Here, in terms of size, looks and apparel, the image could be modifiable, e.g. alienated, by the system or entries into the system. In this embodiment, the body position of the person training is initially recognized by way of sensors and the image is created dependent thereon. Here, it is possible to create a 1:1 image. This can be varied by intended movements that are predetermined by the system. Alternatively, the image of the person training can also be used directly for producing the avatar and this can be enriched by symbols (e.g.: arrows) of an intended movement.

Alternatively or additionally, a visualization unit may be configured to show a static image or a movement of a person, in particular of an avatar. The system is intended to motivate a person training to thus reproduce the position or the movement of the avatar. Feedback means which reward the person training in the case of a successful reproduction or otherwise bring about negative feedback may be provided. By way of example, an EMS in the form of comfortable tingling may be brought about as a reward. A slight “electric shock” can be brought about as negative feedback. In this way, a game situation is facilitated which motivates the person training to put in more effort. A pattern is shown to the person training, which they can try to emulate. This may be carried out in conjunction with a video game, in which it is possible to produce targeted scenes, in which the user must carry out certain movements, such as sword fighting, chopping wood, running, teeing off in golf, a tennis stroke, etc.

In a preferred embodiment, the apparatus can be configured to generate electric energy which is used for EMS. Thus, for example, a stepper, a training bicycle, a rowing machine or any other training appliance may be respectively connected to a generator, and the energy can be extracted thereby. Capacitive energy extraction is also possible, such as e.g. when displacing appropriately equipped training appliances.

For the mobile use, all components preferably have a watertight configuration, or at least such a configuration that water cannot cause any damage. However, this is not necessarily true for an input and output unit which, for example, may be a commercially available smartphone.

A sensor, in particular a strain gauge, may be configured to recognize a posture, such as in particular the angular position of a joint, of a person training with the system or to recognize a movement of a body part or of the entire body of the person training and to bring about electrostimulation depending on the posture, in particular the angular position, or the movement, in particular the speed thereof. In this way, a multiplicity of movements can be carried out. By way of example, a bicep curl movement is possible, as is often demanded within rehabilitation. The electrostimulation can be activated depending on, and in particular proportional to, the aperture angle of the elbow; this is helpful if a person has difficulty with small aperture angles. A stimulation depending on the speed with which the movement is carried out is also possible. Here, the relationship that small movements cause small stimulations and fast movements cause large simulations is possible. By way of example, this is helpful when simulating a swimming movement, where the water resistance increases in the case of high movement speed. These postures and movements can easily be recognized by means of strain gauges. These may be worked into the training apparel or may be adhesively bonded onto the skin of the person training.

In particular, one or more defined sequences of movements, with which the image (avatar) can move, are stored in the system in this case. By way of stimulation pulses, the system can assist or correct the movement of the person training in such a way that the deviation between a carried out movement of the person training and the defined sequence of movements is minimized. Thus, a learning effect of a correct movement is trained, like, for example, for golf. The carried out movement of the person training can moreover be displayed for presenting the deviation of the carried out sequence of movements from the defined sequence of movements. Thus, for example, the avatar may be shown in color and, in the process, the deviations which the person training carries out may be shown using dashed lines or as a gray form/area or the like in the foreground or background.

Further, the system may be configured to present an image of a person training with the system and, moreover, at least one teammate. Here, the teammate may be a real person training, a virtual person, an animal or a fantasy Figure. Moreover, game situations are providable, in which interactions between the images are producible in order to produce electrostimulation depending on the interactions. By way of example, it is possible to represent a sword fight and, if the person training receives a blow, this is acknowledged or confirmed or penalized by means of an electric stimulation. Further, provision can be made of a sensor, in particular a plurality of sensors, in order to record measurement values about the state of a person training with the system and the system is, in this case, configured to adapt, in terms of its movement, the image of the person training to the state of the person training. By way of example, if the pulse or other performance-related states of the person training, either on their own or in combination, assume a value that is too large, the image, i.e. the avatar, can be reduced in terms of the speeds thereof. This avoids a strain on the body of the person training that is too large. This regulation may, in particular, be combined in conjunction with the EMS. The movements of the avatar may be reduced and, likewise, the EMS may be accordingly reduced in a predetermined or variable ratio. Since the person training moves with the movement intensity of the avatar, their movement intensity is reduced. The inverse dependence is also possible, namely that a movement intensity of the person training that is too low increases the movement of the avatar and, at the same time, increases the EMS.

In particular, a ratio for adapting at least two stimulation pulse parameters may be predetermined in the data processing device. In addition, if measurement values of one or more sensors change, the adaptation of these parameters in accordance with this ratio may be provided, wherein the stimulation pulse parameters may be parameters for the same electrode or different electrodes. This should be explained using the following example: by way of example, there is a ratio between the pulse strength (e.g. voltage) of biceps muscles and thigh muscles of 2:1 such that the thigh muscles are activated more strongly. By way of example, if a measurement value that represents the activity of the person training, such as e.g. pulse, respiratory rate or blood sugar value, recedes too much, the activity of these muscle groups can be adapted by way of EMS using the defined ratio. It is possible to fixedly predetermined different ratios for respectively different parameter pairs, or these ratios can be adjustable by the user and/or a trainer.

Moreover, a plurality of sensors may be configured to record different measurement values. Here, different measurement principles may be used in the sensors. The controller or the system is configured to weight these measurement values in a comparison and trigger stimulation pulses herefrom and, in the process, modify stimulation pulse parameters. Thus, for example, a sensor such as a camera can recognize the movement of the person and, moreover, the pulse of the person is measured. Hence, if different sensors respectively record measurement values which, with combined weighting, indicate that an adaptation of the electrostimulation should be carried out, then this is undertaken.

Preferably, the textile electrodes described herein consist of a material which requires no further moisture supply and which draws the required moisture from only the skin moisture produced by the body and the air humidity. To this end, use should be made of a hydrophilic yarn. A mixture of hydrophilic yarn and a synthetic yarn that has been coated in a conductive manner is also possible, as a result of which the additional moisture supply should become superfluous. An EMS apparel may be equipped with at least two electrodes. A textile with two electrodes can be used in a targeted manner for e.g. strengthening the abdominal muscles, the neck muscles or else the muscles of the buttocks. Four electrodes may be used for a simultaneous training unit of two body regions such as abdominal muscles and muscles of the buttocks, back muscles and abdominal muscles, leg muscles and arm muscles, or else any other conceivable muscle combination. It is possible to address any number of muscle groups for as long as channels are provided in the electronics and electrodes for the corresponding muscle groups. An electrode for muscle stimulation should have an application area that is as large as possible in order to ideally conduct the current up to the muscle. The larger the application area, the more comfortable the current transfer is as well. The size of the electrode should be adapted to the body parts and the relevant muscle groups, meaning that electrodes for the abdominal muscles should be greater than electrodes for the chest muscles.

In the best case, an electrode consists of a conductive material and requires no further supply of an additional substance such as gel or water. Said electrode may consist of a silver-coated synthetic yarn which is coated with a polyelectrolyte film. If it should nevertheless be the case that an additional conductive means is required, the electrode may be applied to a carrier material which keeps moisture and therefore contributes to the conductivity. By way of example, this can be a silver electrode that has been embroidered onto moisture-storing neoprene.

The invention also provides for an apparel piece for electrostimulation comprising at least one first portion (A) and at least one second portion (B), wherein portion (A) has a component of conductive yarns and/or threads and/or fibers of 12 to 100%, portion (B) has a component of conductive yarns and/or threads and/or fibers of 0 to 11° A, wherein the component of conductive yarns and/or threads and/or fibers in portion (A) is always greater than the component of conductive yarns and/or threads and/or fibers in portion (B) and the transitions from portion (A) to portion (B) are seamless over at least 75% (in relation to the overall length of all transitions). In the apparel piece, the transitions from portion (A) to portion (B) may be completely seamless and/or the entire apparel piece may be seamless. In respect of its area, the apparel piece preferably has 2 to 40% portion (A) and 20 to 98% portion (B). The apparel piece is preferably produced in a round knitting method.

When this description refers to a “textile”, this can mean, depending on context, either an apparel piece or a textile construct, produced from yarn or thread, such as wovens, knits, braids, stitch bondeds, nonwovens and felts.

The present invention also relates to a functional apparel with a tactile stimulus module and EMG electrode. The functional apparel is an embodiment of the above-described textile or apparel piece. The tactile stimulus module is an embodiment of an above-described electrode and the EMG electrode is an embodiment of an above-described sensor.

In addition to the aforementioned objects, the apparatuses as described below also achieve the object of providing systems which facilitate collecting information items about the wearer of the systems and transmitting information items to the wearer of the systems; in particular, this relates to a protection system for dangerous situations. It is a further object to provide a system which facilitates the wearer to learn complicated sequences of movements, in particular with the interaction of a trainer.

This object is achieved by a functional apparel which is configured and embodied to be worn on the human body, comprising at least one contact unit with a contact face which is embodied and configured for direct contact with the skin surface of a user, wherein the contact unit has a tactile stimulus module and a first sensor, wherein the first sensor represents or comprises an EMG electrode for measuring and/or detecting the electrical muscle activity, further comprising an energy storage apparatus, which supplies the tactile stimulus module with power, and a first internal data processing device, which has an operative connection to the EMG electrode and/or the tactile stimulus module.

Surprisingly, it was found that a functional apparel is particularly suitable for housing contact units with tactile stimulus modules and EMG electrodes. Functional apparels within the meaning of the present invention are materials which surround the body of the human as an artificial envelope with a more or less tight fit, with additional functions, for example the measurement of EMG signals in the present case, being present. By way of example, these include appropriately equipped armbands, shoes, gloves, headgear, belts, shirts, trousers, socks and jackets. Preferably, the functional apparel is an apparel piece which is selected from the group consisting of armbands, suits, shirts, jackets, blouses, T-shirts and trousers. In particularly suitable configurations, the functional apparel is a suit, in particular a suit which covers arms, legs and the upper body.

It should be clarified that if the contact unit comprises a tactile stimulus module and a first sensor, this does not preclude the contact unit from comprising more than one stimulus module and/or more than one first sensor. Analogously, the first sensor may also comprise more than one EMG electrode. In a preferred configuration, the respective contact unit comprises exactly one tactile stimulus module and exactly one first sensor, and so the number of tactile stimulus modules and first sensors per contact unit is 1 in each case.

Preferably, provision is made for the first internal data processing device to be designed and configured to control the tactile stimulus module and/or the energy storage apparatus on the basis of the sensor information items from the first sensor. Here, provision may be made in one configuration for the data processing device to register a muscle being stretched by means of the first sensor and thereupon control the energy storage apparatus to the effect of the energy storage apparatus transmitting an energy pulse to the tactile stimulus module.

In a suitable configuration, provision is made for the functional apparel to comprise a first textile, wherein the contact unit is connected to the first textile, in particular sewed or adhesively bonded to the latter. In a preferred configuration, the first textile is the textile which predominantly forms the functional apparel, i.e. by more than 50% by weight, in particular by more than 75% by weight thereof.

The connection between the contact unit and the first textile may have a detachable or non-detachable embodiment. Should the contact unit have a secure or non-detachable connection to the first textile, in particular by way of sewing or adhesive bonding, it was found that it is easier to house the contact unit without the latter disturbing the user during the movement. This also is advantageous in that the contact unit cannot be inadvertently detached. If the contact unit is connected to the first textile in a detachable manner, the aforementioned is rendered more difficult, but detachability is advantageous in that a replacement of contact units, in particular for servicing, cleaning and/or repair, is possible in a particularly simple manner. Also, cleaning the functional apparel is significantly simplified. Particularly in the case of the requirements of a washing procedure at an elevated temperature, it may be advisable to remove possibly present contact units from the apparel. In a particularly preferred configuration, the connection, in particular the detachable connection, between contact unit and functional apparel is achieved by virtue of the first textile having a fastening means, in particular a hook-and-loop fastener or a snap fastener, by means of which the contact unit is connected.

According to the invention, configurations are particularly preferred if the functional apparel is autonomous. Autonomous within the meaning of the present invention means that the functional apparel, in particular the contact unit of the functional apparel, does not require any permanent wired connection to external energy storage apparatuses or data processing devices for the functionality thereof, in particular in view of the stimulus transfer and the EMG measurement. However, this does not preclude the functional apparel from being temporarily connected by a cable, for example in order to recharge the energy storage. In preferred configurations, the present functional apparel is also autonomous to the extent that a wireless communication with an external data processing device is not required for the functionality of the tactile stimulus module and/or the EMG electrode. Preferably, this is a functional apparel which has an offline mode and an online mode, with communication with an external data processing device, in particular a smartphone, only occurring in the latter. Preferably, provision is made for an activation of the stimulus modules, in particular by way of the first internal data processing device, being possible even in the offline mode. In the present case, an activation of the stimulus modules is understood to mean that the stimulus modules trigger a stimulus.

In an expedient configuration, the contact units are also configured autonomous in each case and, in particular, each have a dedicated energy storage apparatus and may, in the process, communicate wirelessly, preferably with the first internal data processing device.

Reference is made to the fact that if statements are made about a unit, for example “a” sensor, “an” energy storage apparatus, “a” data processing apparatus, “a” first contact unit, this does not preclude more than one unit, for example a plurality of first contact units, from being present. In many embodiments, this even preferably is the case.

External energy storage apparatuses and external data processing devices within the meaning of the present invention are apparatuses and devices which are not constituent parts of the functional apparel according to the invention. An external data processing device within the meaning of the present invention may be, for example and preferably, a smartphone which wirelessly communicates with the first internal data processing device.

In particular, it is preferable for the functional apparel to have contact units which are arranged in such a way that these come to rest on different body parts by way of their respective contact faces in the case of the generic wear of the functional apparel. In a preferred configuration, the functional apparel comprises at least four, in particular preferably six and very preferably at least 10, spatially separated contact units. In some configurations, provision can even be made for a multiplicity of contact units, in particular more than 20 or 40 contact units, to be contained within the functional apparel. In one configuration, the functional apparel comprises a network of first sensors and/or contact units, wherein at least some, in particular the majority and/or all contact units are connected to one another in a wireless or wired manner. Here, the connection can be direct or indirect. Numerous sensors, in particular a network of first sensors, facilitates more accurate monitoring and optionally controlling of muscles. Consequently, it is possible to exactly register which muscle is tensioned. Particularly when transferring complex sequences of movements, it was found that conventional apparatuses are not suitable for this to the same extent.

A tactile stimulus module within the meaning of the present invention is a module which transmits tactile stimuli to the body part on which the module rests. Here, this may relate e.g. to electrical or mechanical stimuli. It is likewise conceivable for the module to increase its temperature in order to trigger a stimulus on the body part. The body part is preferably the skin surface, on which the module rests, and/or a muscle which is situated under said skin surface.

An expedient embodiment of the invention provides for the at least one tactile stimulus module to comprise or represent an EMS electrode for the electrostimulation of muscles. Here, provision can be made for the tactile stimulus module to transfer current pulses, in particular current pulses that are controlled by the first internal data processing device. Preferably, the EMS electrode of the tactile stimulus module is an electrode which is connected to the contact face of the contact unit or formed by the latter.

In a further configuration, the tactile stimulus module is a vibration module. Such a configuration is advantageous in that a vibration module does not significantly impair the measurement of the EMG electrode, particularly while the measurement is carried out, if there is no vibration. It was also found that users often perceive vibrations to be more comfortable than current pulses. By contrast, a disadvantage in relation to current pulses consists in the slower reaction to the vibrations. The muscle is influenced directly in the case of current pulses. For this reason, the present invention, in all possible configurations thereof, also comprises embodiments in which other tactile stimuli such as vibrations are generated at the user by way of the apparatus or the system instead of EMS pulses.

In a suitable embodiment, provision is made for the first internal data processing device to comprise a first transmission module and/or be connected to a first transmission module by wires, wherein the first transmission module is preferably designed and configured to communicate with an external data processing device, in particular a smartphone. Such a wireless communication option between the first internal data processing device and the external data processing device may allow the first internal data processing device to receive information items. By way of example, these can be instructions by the external data processing device relating to activating the tactile stimulus module. Alternatively or additionally, the wireless connection option preferably allows settings of the first internal data processing device to be modified. A transmission module within the meaning of the present invention is preferably a module which is designed and configured to transmit, and preferably also receive, data, in particular digital data, using electromagnetic radiation.

In a further configuration, the first internal data processing device facilitates a wireless communication with the contact unit, the EMG electrode and/or the tactile stimulus module by way of the first transmission module or a second transmission module of the first internal data processing device. In particular, it is preferable for the first internal data processing device to have a wireless operational connection to the EMG electrode and/or the tactile stimulus module, with the contact unit preferably having a third transmission module for this purpose.

Preferably, the third transmission module is designed and configured to communicate with the first and/or second transmission module. Here, it is particularly preferable for the contact unit to comprise a third transmission module. Here, provision can be made for the first internal data processing device to evaluate data, which are received by the EMG electrode, and, on the basis of these data, produce instructions for the tactile stimulus module, which are transmitted wirelessly in each case.

What is considered in an alternative configuration is that the first internal data processing device has a wired operative connection to the EMG electrode and the tactile stimulus module. In contrast to the wireless variant described above, the EMG electrode and the tactile stimulus module can only be placed at positions which admit a wired connection. Further, wired conveying options are disadvantageous in that the wires have a bothersome effect within a functional apparel, for example by virtue of restricting the freedom of movement and/or by virtue of the wires constituting an injury risk. Nevertheless, it was found that a wired connection is advantageous in that the data transfer is interrupted less often than in the case of wireless connection options.

In further configurations, provision is made for the functional apparel to be designed and configured to be worn on the human upper body, in particular representing or comprising a whole body suit, a jacket and/or a vest, and/or that the functional apparel is designed and configured to be worn on human arms or legs, in particular representing or comprising trousers or an armband, and/or that the functional apparel is configured and designed to be worn on the feet or hands, in particular representing or comprising shoes, socks or gloves, and/or that the functional apparel is designed and configured to be worn on the head, in particular representing or comprising headgear. Apparel pieces which at least partly cover the arms and/or legs were found to be particularly suitable. In particular, it is preferable if the functional apparel is designed and configured to enclose at least one body part.

A whole body suit within the meaning of the present invention is a suit which, when used as intended, predominantly covers a human body, i.e. more than 50%, in particular more than 75% thereof. In the process, the arms and legs and the torso, in particular, are substantially completely covered. It is preferable if the feet and hands are also covered by the whole body suit. In a particularly preferred configuration, provision is made for a whole body suit also to cover the head such that the human body is covered not only predominantly, but completely. Preferably, a whole body suit has an integral embodiment.

In a particularly suitable configuration, the functional apparel covers both arms and both legs at least in part, in particular predominantly. Predominantly within the meaning of the present invention means more than 50% of the overall area of the affected body parts. Preferably, predominantly means more than 75%, in particular more than 80%. In a very particularly preferred configuration, the functional apparel is a suit, in particular an integral whole body suit. It was found that the functional apparel which predominantly covers body parts ensures a particularly secure hold of the contact unit, in particular of the contact face, on the skin of a user. In so doing, it is even possible to carry out athletic activities without the contact face slipping.

Preferably, the functional apparel comprises at least two contact units, wherein the at least two contact units are preferably available in or at the functional apparel in such a way that said contact units come to rest on opposite body regions when the functional apparel is worn. In a suitable configuration, this relates to opposite body regions of the head, of the neck, of the torso, in particular of the shoulders, and/or opposite extremities, in particular opposite arms and/or legs. Here, it is particularly expedient if the at least two contact units on the human body come to rest on opposite shoulders, arms and/or legs.

Surprisingly, it was found that tensions, which occur during office routine, can be reduced by a stimulus on the contralateral or opposite side. Here, the EMG electrode of a contact unit measures an increased muscle activity on one side and a stimulus module of a contact unit arranged on an opposite body part is thereupon activated, in particular by way of an internal or external data processing device. This causes relaxation.

In a further configuration, at least one contact unit comes to rest on the spinal column of the human body when the functional apparel is worn generically. This renders it possible to measure signals of the brain at the spinal column and influence the spinal column, optionally directly by way of current pulses. In particular, it is possible to at least partly inhibit the transfer of pain signals.

It was found that two contact units or the symmetric arrangement of the contact units is connected with a significantly improved stimulus transfer onto the human body.

Further, provision is made in some configurations for the functional apparel to comprise at least one second sensor in addition to the first sensor, said second sensor being selected from a group consisting of IR sensor, ultrasonic sensor, magnetoresistance sensor, moisture sensor, elongation sensor, temperature sensor and capacitive sensor. This second sensor allows further information items to be collected about the body part on which the contact face is applied. Preferably, the second sensor has an operative connection, in particular a wireless operative connection, with the first internal data processing device such that the latter can evaluate the data from the second sensor or transfer said data to the external data processing device, wherein the data of the second sensor is used to determine when and/or for what time duration the tactile stimulus module is activated.

Alternatively or additionally, provision can be made according to the invention for the functional apparel to comprise at least a third sensor selected from a group consisting of a pulse sensor, respiratory rate sensor, blood oxygen sensor, blood sugar sensor, lactate sensor and heart rate sensor. This third sensor can be used analogously to the second sensor and can communicate analogously to the second sensor with the first internal data processing device.

In particular, it is preferable for a plurality of first, second and/or third sensors to be present such that measurement values which are determined at the contact face can be compared to one another. Further, the second and third sensors facilitate adapting the activity of the tactile stimulus modules to the individual physical strain of the user. Also, particularly in the case of a functional apparel in the form of a protective suit, it is advantageous if these information items are determinable by first, second and/or third sensors such that suitable countermeasures can be introduced by means of the tactile stimulus module or by transmitting warning notices or emergency information to the external data processing devices.

Preferably, the contact face comprises a second textile, in particular a second textile that is conductive at least in sections. In particular, provision is made in a particularly preferred configuration for the tactile stimulus module and/or the EMG electrode to be constituent parts of the second textile or to be embodied by the second textile. In a further configuration, provision is made for the contact face and/or the tactile stimulus module to comprise the aforementioned, preferably conductive second textile. Conductive textiles can be used to transfer current pulses onto a skin area or measure the muscle activity by means of the EMG electrode. It was found that an effective transfer of current pulses is possible, with the conductive textiles facilitating an adaptation to the skin structure. Further, conductive textiles can be incorporated better into existing textiles, in particular the first textile, of the functional apparel.

It is preferable for the conductive second textile to at least partly embody an electrode or be a constituent part of an electrode, in particular the EMG electrode and/or the EMS electrode. This ensures that the supplied current is distributed uniformly in the electrode. Preferably, the conductive second textile is a constituent part of the contact face or forms the latter. In an expedient configuration, provision can also be made for the EMG electrode and/or the EMS electrode to comprise the contact face or to be a constituent part thereof.

In a preferred configuration, the electrode, in particular the EMG electrode and/or EMS electrode, has a cushion placed under it. This is advantageous in that additional contact pressure is produced for the contact face and/or for the above-described electrode. This is expedient, particularly in the case of concave body regions.

Preferably, the EMG electrode and/or the EMS electrode is an EMG electrode pair and/or an EMS electrode pair. Preferably, use is made of an electrode, in particular the EMG electrode and/or the EMS electrode, which has a first contact face and a second contact face, wherein the first contact face is preferably smaller than the second contact face. Here, it is preferable if the circuit is designed and configured in such a way that the entry of the current into the body respectively occurs via the first contact face and the exit occurs via the second contact face.

A particularly preferred EMG electrode comprises an outer ring electrode and an inner ring electrode. Here, the outer ring electrode preferably forms the second contact face and the inner ring electrode forms the first contact face.

Further, it is preferable if the contact unit is connected or connectable to the first internal data processing device and/or the external data processing device in a wireless and/or wired manner. This is advantageous in that data recorded by the contact unit are able to be evaluated by the respective data processing device and instructions are able to be transmitted from the respective data processing device to the contact unit.

Preferably, the functional apparel is able to emit signals via acoustic and/or visual signaling devices. In a particularly suitable configuration, this relates to a band which is worn over the back of the hand and which comprises said signaling devices, in particular LED lamps. Preferably, the band is fastened by means of a loop on the thumb.

Preferably, the data processing device comprises a microcontroller which is designed and configured to control the at least one tactile stimulus module, in particular the signal frequency, signal intensity, signal duration thereof and/or the signal spacing of signals which are emitted by the at least one tactile stimulus module. Preferably, the signals are electrical pulses or vibrations.

The following explanations in respect of the electrodes relate to the EMG electrode and/or the EMS electrode, as described above, provided that nothing else is said.

Electrodes which comprise a first yarn, in particular which consist of the latter, are particularly preferred. Preferably, the first yarn is conductive. It was possible to show that flexible electrodes impair the free movement of the body part, in particular during an athletic exertion of the user, only to a small extent. Further, the risk of injury is lower in the case of flexible electrodes than in the case of rigid electrodes made of solid metal components.

Preferably, the electrodes comprise a first yarn or consist of same, wherein the first yarn is coated or encased by a first metal or the compound of a first metal. In particular, it is preferable for the compound of the first metal to be a metal oxide, preferably a first yarn which is coated with the metal oxide of the first metal. The aforementioned compound is a chemical compound, preferably a covalent chemical compound or an alloy. Said coating or jacket is preferably less than 1 μm, in particular less than 0.1 μm, preferably less than 0.01 μm thick.

In an embodiment, this may also relate to a first yarn which has been mixed with a second metal or the compound of a second metal which, in particular, is not identical to the first metal. In a further configuration, it is also preferable if use is made of a first yarn which has been mixed with metal threads, i.e. very thin metal wires. Preferably, the first yarn has been mixed with metal threads which consist of a pure second metal and are additionally coated with a metal compound, in particular an oxide of the first metal.

In a configuration, the first yarn comprises the second metal or a compound of the second metal and/or said first yarn is coated with the first metal or the compound of the first metal, wherein the first metal and/or second metal is selected from the group consisting of silver, copper, titanium, gold, aluminum, zinc, and iron. A coating or jacket of titanium or a titanium compound, in particular titanium oxide, is particularly preferred.

Further, it is preferable if the electrode comprises silicone, in particular if said electrode is multilayered, wherein at least one layer consists of silicone or comprises the latter. Silicone is particularly skin friendly. Here, it was found that a contact face which comprises silicone is particularly suitable as an electrode for the EMG electrode and/or the tactile stimulus module. Instead of using a smooth surface, it is preferable for the surface to be structured and/or to have present a form adapted to the anatomy of the body. The silicone electrodes can be multilayered, in particular consist of a non-conductive layer and a conductive layer. In order to increase the tensile strength, the silicone electrodes preferably contain an integrated third textile or are connected to the second textile, wherein the textiles preferably have a coefficient of expansion that corresponds to that of the employed silicone.

In particular, it is preferable if use is made of a conductive polymer composition, preferably a conductive silicone composition. Here, this may be e.g. a silicone composition, which was mixed with metals and/or metal compounds. Instead of modifying the silicone composition itself, provision can also be made of introducing metallic components, in particular wires, into conventional silicone.

In particularly suitable configurations, an electrode pair forms the tactile stimulus module in the form of the EMS electrode, and also the EMG electrode. Here, the electrode pair satisfies a dual function, wherein, at a first instant, the EMG measurement is carried out with the EMG electrode and, at another, second instant, electrical pulses are transmitted to the EMS electrode. In a further embodiment, the EMG electrode and the tactile stimulus module are arranged spatially apart, but preferably adjacent to one another.

Preferably, the functional apparel comprises an areal energy production element, in particular a flexible solar module. In a preferred embodiment, the functional apparel comprises an energy-producing second yarn, which preferably forms the areal energy production element or which is a constituent part of same, which in particular forms the first textile, second textile and/or a third textile or is a constituent part thereof.

In a preferred embodiment, this relates to a second yarn which obtains energy during deformation as a result of the piezoelectric effect and which comprises a piezoelectric material. Preferably, the second yarn comprises a metal oxide. In a particularly preferred configuration, this relates to nanowires made of said metal oxide, which are preferably aligned radially in relation to the longitudinal direction of the second yarn.

In a further preferred embodiment, the second yarn absorbs sunlight and converts the latter into electric energy, in particular by means of solar cells, preferably by means of textile solar cells. Particularly preferably, use is made of an encased metal wire made of a third metal, which is encased by a fourth metal or a compound which contains a fourth metal. The third metal and fourth metal are preferably selected from a group consisting of silver, copper, titanium, gold, aluminum, zinc, and iron. Preferably, the third metal and fourth metal are not identical, with it being particularly preferred for the conductivity of the third metal to be higher than the conductivity of the fourth metal or the compound with the fourth metal. In particular, it is preferable for this to have deposited on it a compound with a fifth metal, with this compound preferably having a perovskite crystal structure.

Preferably, the second yarn comprises a semiconductor material, in particular a textile semiconductor material. In an expedient further configuration, the second yarn comprises powder-coated nanotubes.

It could be shown that the use of a second yarn and/or an areal energy production element renders it possible to charge the energy storage in a wireless or wired manner. The wireless charge can be carried out by way of an induction element. In particular, it was found that the second yarn or an areal energy production element is less bothersome to the user of the functional apparel than a conventional box-shaped energy storage.

Preferably, the functional apparel comprises a third yarn which is able to store energy, which, in particular, is a constituent part of the energy store or which forms the latter. It could be shown that flexible capacitors are suitable for storing energy and forming the third yarn. In an embodiment, the third yarn comprises particles made of activated carbon. It could be shown that the functional apparel needs to be supplied with energy from an external energy source less frequently if use is made of such a third yarn.

Preferably, the functional apparel comprises at least one contact sensor, at least one proximity sensor, and/or at least one temperature sensor, preferably at least one contact sensor. In particular, it is preferable for the functional apparel to comprise a sensor area which has at least one contact sensor, at least one proximity sensor, and/or at least one temperature sensor. In particular, it is preferable for the contact sensor, proximity sensor, and/or temperature sensor to be connected to the internal data processing apparatus and/or external data processing apparatus in a wireless or wired manner.

In an expedient configuration, the at least one contact sensor, at least one proximity sensor, and/or at least one temperature sensor, in particular the sensor area, are present on the outer side of the functional apparel during generic wear, with the outer side being the side facing away from the body. It could be shown that the user of the suit can influence the latter in a targeted manner by way of approaching or touching said sensor with their hand. Particularly preferably, this relates to a sensor area that is designed and configured to transmit instructions of the user to the internal first data processing device or internal second data processing device or to the external data processing device.

Preferably, the at least one contact sensor, at least one proximity sensor, and/or at least one temperature sensor, in particular the sensor area, are present on the forearm or the hand, in particular on the upper side and/or lower side of a segment of the functional apparel surrounding the forearm, in the case of generic wear. The upper side and lower side of the surrounding element correspond to the position of the upper and lower side of the forearm during generic wear of the functional apparel. It could be shown that this arrangement facilitates largely unimpeded operation.

In an expedient configuration, the functional apparel comprises a display, in particular a display which is a constituent part of the sensor area or embodies the latter. Here, this preferably relates to a touch-sensitive display, in particular a bendable touch-sensitive display. A flexible display which can be deformed is particularly preferred. Such a display is preferred, particularly in the case of protective suits.

In a further embodiment, a plurality of first sensors are comprised in the functional apparel, said first sensors preferably communicating in a wireless or wired manner, in particular wherein a plurality of contact units are comprised, each of which comprise a first sensor. This is advantageous in that comprehensive information items about the activity of various muscles can be collected by the network of first sensors and also that stimuli can be transferred highly selectively to specific body regions.

In a further configuration, provision is made for the functional apparel to comprise a plurality of electrodes which are connected via conductor tracks that have been worked into the first textile and/or second textile of the functional apparel. Here, this may be the EMG electrode and/or the EMS electrode of the tactile stimulus module.

In an expedient configuration of the functional apparel according to the invention, provision is made for the at least one tactile stimuli module and the at least one first sensor to have an operational connection or be able to be brought into an operational connection by way of the first internal data processing device, wherein, preferably, the control signals for the tactile stimulus module that are based on the data or measurement values of the at least one first sensor are producible or produced by way of the first internal data processing device.

In a preferred configuration, provision is made for the first data processing device to be integrated into the contact unit, in particular for said first data processing device to be a constituent part of the contact unit. This facilitates the decentral provision of data processing devices for each contact unit, and so a long distance connection between the contact units is not required.

According to the present invention, it is particularly preferred if the first sensor is present adjacent to the tactile stimulus module. This is advantageous in that muscle activity is determined at substantially the same location as the location at which the tactile stimulus is also transferred. Hence, it is possible, for example, to transmit a sequence of movements via the tactile stimulus module, with the first sensor determining, at the same time, whether the transmitted sequence of movements has also in fact been carried out.

In a configuration, provision is made for the contact unit to comprise a casing, in which the first sensor, the tactile stimulus module, the first transmission module, and/or the first internal data processing device are housed. In a preferred configuration, at least the internal data processing device and the first transmission module are housed in the casing.

In a further embodiment, the electrodes comprise a hydrophilic yarn, in particular a first yarn which is preferably a constituent part of the second textile. Alternatively or additionally, the electrodes may be provided with a moisture-providing layer that lies between the skin and the conductor. The aforementioned measures improve the conductivity between the electrode and the skin. It was possible to show that the reliability of the EMG measurement and the transfer of EMS pulses is significantly improved.

Further, it is preferred if the functional apparel represents a suit, in particular a single-piece suit, or comprises the latter. By way of example, this can be a track suit. Using a suit, it is possible to provide instructions, which are transmitted in the form of stimuli, depending on the information items obtained by the sensors (first sensor, second sensor, and/or third sensor). Hence, a trainer can teach sequences of movements by virtue of transmitting stimuli which should introduce the sequences of movements and said trainer can subsequently monitor, by means of said sensors, whether the sequences of movements have in fact also been carried out correctly. What may be important here is that second and/or third sensors, as described above, are available so that the trainer can evaluate whether there is an overload. In this case, he can interrupt the training or adapt it in such a way that the strain lies in a normal range.

In particular, it is preferable for the functional apparel to represent or comprise a diving suit or astronaut suit. It was recognized that there is a particular need for contact units, as were described above, in the case of astronaut suits. At their usual place of work, astronauts move in weightless conditions, i.e. they are not, or only slightly, subject to the influence of gravity. This represents a significant change in relation to the stimulus surroundings on the Earth's surface. This can be at least partly counteracted by way of an appropriate activation of the tactile stimulus module. Further, astronauts often have to undertake repair works in space. Here, they receive instructions from Earth, which have to be carried out. In this respect, the astronaut suit according to the invention renders it possible to transfer instructions for sequences of movements via stimulus modules. In particular, under the conditions of space, this represents assistance that cannot be underestimated. A diver is also subject to similar conditions as an astronaut. Said diver also moves in a type of weightlessness and has to receive instructions, for example in the case of repair works on offshore platforms, and so assistance with an appropriately modified diving suit is possible in a manner analogous to the astronaut suit.

Preferably, provision can also be made for the functional apparel to be or comprise a protective suit, in particular a protective suit in relation to a temperature gradient, preferably a neoprene suit. This embodiment is preferred, particularly in combination with a tactile stimulus module which is able to trigger vibrations. By way of example, it is conceivable that the suit activates muscles in order to produce heat should hypothermia be impending so as to provide the person with time until the danger has receded or until rescue. Moreover, such a suit facilitates monitoring of vital functions, the data of which can, for example, already be transferred wirelessly in advance to rescuers via the external data processing device.

In a further configuration, the functional apparel comprises at least one thrombosis stocking or represents the latter. This embodiment can be used during long flights for thrombosis prophylaxis by virtue of the muscles in the lower extremities being stimulated during the flight. To this end, the electrodes are preferably worked into the stockings or cuffs. The information from the measurements by the sensors can be used here to regulate the tactile stimulus module or the tactile stimulus modules.

Further, it is preferable for the functional apparel to have a multipart configuration and, in the process, comprise goggles, preferably with a display. In particular, it is preferable if this relates to virtual reality goggles with a display. Here, provision can preferably be made for the goggles to have the first internal data processing device or comprise a second internal data processing device, with the second internal data processing device being designed and configured to communicate with the first internal data processing device, the contact units and/or the external data processing device. The above embodiment facilitates feedback between the user and the virtual world or the avatar (e.g. a virtual trainer) of the virtual world. By way of example, if one is hit by ammunition within the scope of a computer game, there may be haptic feedback, e.g. a vibration signal or an electric pulse.

It is preferred in a further configuration if the contact face forms or comprises an electrode or is a constituent part of same. Preferably, this is the EMG electrode and/or the EMS electrode, particularly preferably both the EMG electrode and the EMS electrode. Further, it is preferable if the contact face comprises or represents the tactile stimulus module or is a constituent part of same, particularly if this relates to a tactile stimulus module in the form of an EMS electrode. It is also preferable for there to be at least one direct connection to the stimulus module, even if the tactile stimulus module is not part of the contact face. Direct preferably means that the stimulus module has direct contact with the contact face.

It is particularly preferable for the display to be an LED display, in particular an OLED display which is controlled by the second internal data processing device, with the second internal data processing device being designed and configured to control the tactile stimulus modules.

Further, a system for suppressing pain, comprising a functional apparel as described above, is a subject of the invention. Here, the stimulus module is activated in the case of pain which, for example, is determined by the first sensor. In one embodiment, said stimulus module transfers electrical stimuli which ease the pain. The assumption is made that electrical signals are sent to the spinal cord by the stimulus of nerves lying in the tissue, said electrical signals damping the signal propagation of the pain and/or causing the release of chemical substances in the brain which reduce the perception of pain. A stimulus module which exerts pressure or emits heat can also reduce the pain. In end effect, the stimulus (which is not painful or less painful) triggered by the stimulus module is overlaid onto the stimulus that is perceived as painful and that should be counteracted. Stimulus paths of the central nervous system for forwarding the painful stimulus from the periphery to the brain are influenced in such a way in the process that the pain propagation to the brain is reduced or prevented. The system is suitable for both acute and chronic pain. However, in the present case, a system is provided, in particular in conjunction with musculoskeletal pain, such as e.g. back pain, said system being suitable, in particular by means of the first sensor, to recognize the regions in which and/or the times at which active pain suppression is required. This may be advantageous, particularly in the case of patients who cannot communicate and/or who are not under constant care.

The present invention provides a functional apparel which can transfer stimuli to body parts. In one embodiment, electrical pulses are transmitted to muscle groups, as a result of which the muscle or the muscles perform a contraction. Here, an individual adaptation of the electrical pulses or the vibrations to the locally present muscular strain is possible. In a further embodiment, vibrations are transferred to the skin surface such that the latter experiences a stimulus and, preferably, is moreover heated. Numerous fields of application are conceivable here. By way of example, the functional apparel can render it possible to transfer instructions relating to a sequence of movements to the user. This allows athletes to learn specific, difficult sequences of movements intuitively on the basis of directly perceived stimulus stimulations. Here, a trainer can transfer instructions with the aid of the present apparel and, at the same time, track whether said instructions are followed. It is also conceivable that instructions can be transferred in dangerous situations. Here, the transfer of electrical pulses may, in some configurations, ensure that the muscles can be controlled in accordance with the instruction orders (independently of the deliberate control of a body movement by the user). This may be advantageous, particularly in dangerous situations which require a particularly fast reaction. Moreover, it is a particular concern of the present invention to be able to transfer stimuli in virtual surroundings, depending on the muscle activity of the user. Here it is possible, for example, for a virtual avatar in virtual surroundings to simulate muscle contractions that are analogous to those in fact carried out by the user. It is also possible to simulate stimuli of the virtual world by way of a stimulus transfer by means of the tactile stimulus modules. By way of example, if a player hits a virtual table edge with their arm, muscular contraction or vibration may be caused at their arm.

Consequently, a device or a system is part of this invention, which represents functional apparel to be worn on the human body, comprising at least one contact unit with a contact surface, is designed and configured for direct contact with the skin surface of a user, with the contact unit comprising a tactile stimulus module and a first sensor, the first sensor representing or comprising an EMG electrode for measuring and/or detecting the electrical muscle activity, further comprising an energy storage apparatus which supplies the tactile stimulus module, in particular the tactile stimulus module and the first internal data processing device, with power, and a first internal data processing device which has an operative connection to the EMG electrode and/or the tactile stimulus module. The first internal data processing device can be designed and configured to control the tactile stimulus module and/or the energy storage apparatus on the basis of data, in particular measurement signals, from the first sensor. The functional apparel may comprise a first textile, with the contact unit being connected, preferably detachably connected, to the first textile, in particular wherein the functional apparel consists predominantly of the first textile. The contact unit may be sewed or adhesively bonded to the first textile and/or connected by way of fastening means, in particular a hook-and-loop fastener or a snap fastener. The at least one tactile stimuli module may comprise or represent an EMS electrode for electrostimulation of muscles and/or the tactile stimuli module may comprise or represent a vibration module. The first internal data processing device may comprise a first transmission module or may be connected to a first transmission module in a wired manner, with the first transmission module preferably being designed and configured to communicate with an external data processing device, in particular a smartphone. The first internal data processing device may comprise a transmission and reception module, in particular the first transmission module and/or a second transmission module, which facilitates the wireless communication with the contact unit, the EMG electrode and/or the tactile stimulus module, in particular in such a way that the first internal data processing device has a wireless operative connection with the EMG electrode and/or the tactile stimulus module. The contact unit may comprise a third transmission module which is preferably configured and designed to communicate with the first transmission module and/or second transmission module. A functional apparel comprising at least two contact units is also within the scope of the invention, wherein the at least two contact units are preferably present in or at the functional apparel in such a way that said contact units make contact at opposite body regions when the functional apparel is worn. The at least two contact units may make contact on the human body on opposite body regions of the head, of the neck, of the torso, in particular of the shoulders, and/or on opposite extremities, in particular the arms and/or legs. The at least two contact units make contact on the human body on opposite shoulders, arms, and/or legs. In the case of the generic wear of the functional apparel, at least one contact unit may make contact on the spinal column of the human body. The contact unit may comprise a casing which preferably encompasses the first sensor, the tactile stimulus module, the first data processing device, the first transmission module, the second transmission module, and/or the third transmission module.

A further part of the invention relates to a suit (and a method) comprising one sensor and/or a multiplicity of sensors, said suit being connected to a virtual world by way of an interface (goggles, helmet, visor, contact lens, display that is situated in front of the eyes or any conceivable embodiment and combination). The data can be transferred offline and/or online; the user receives haptic signals in particular by the suit. Said user may select a course online and/or offline in a virtual training studio, as imparted through the goggles, helmet, visor (visual instrument and sensory organ replacement), the contact lens, or the display situated in front of the eyes. Once a decision about a course has be made, said user starts the virtual course, where they are greeted by an avatar. The avatar/trainer (virtual trainer) demonstrates an exercise and the user simulates it. By way of sensors in the suit, the system recognizes how fast a given extremity is moving, where it is and whether it is moving correctly in space. In this method, the body is preferably subdivided into a vertical main body axis and two horizontal axes, firstly the axis of the shoulders and secondly the axis of the pelvis. The upper extremities are at the axis of the shoulders and the lower extremities are at the axis of the pelvis. By way of appropriate algorithms in software, the virtual model can be compared to the user by way of sensors in the suit, and individually trained by the avatar (virtual trainer), in particular by way of haptic feedback, but also by way of acoustic or optical feedback. There can also be a training via EMS (electrostimulation training) in the virtual world; the avatar demonstrates the exercises and a program then triggers a muscle-stimulating stimulus. If the exercises were taken up correctly, the user receives a stimulation stimulus (training stimulus).

In a further embodiment, a movement form is demonstrated (e.g. a golf swing and all conceivable athletic exercises and movements), and then simulated by the user. By way of the sensors in the suit, the program running in the background recognizes which muscles are active and which are not. Said program is then able to actuate individual muscles during the exercises in order to allow the user to learn or improve a movement. A further exemplary embodiment is that of wearing a suit in space (space station), in order to stimulate the nerves by a pulsating, rising signal, from bottom to top, so as to simulate the innervation behavior on earth. Also, an astronaut may wear this specific clothing in order to monitor and/or stimulate the body.

In the case of an EMS system, one or more defined sequences of movement are stored for the image of the person training, such as, in particular, a golf swing movement. In addition, the system is configured to assist or correct, by way of electrostimulation, the movement of the person training such that the deviation between a movement carried out by the person training and the defined sequence of movement is minimized. In particular, a region or corridor of admissibility of the movements is defined and the system is configured to produce stimulation pulses only if the movement of the person training has departed from the region or corridor of admissible movements.

An even further part of the invention relates to a method that can be used for the PMR (progressive muscle relaxation) method according to Edmund Jacobson. The method serves for deliberate and conscious relaxation and/or tensioning of specific muscle groups, as a result of which a state of deep relaxation of the entire body should be obtained. Here, the individual muscle groups are initially tensioned successively in a specific sequence, the muscle tension is briefly held and the tension is subsequently released. Here, the concentration of the person is directed to the change between tension and relaxation and the sensations that accompany these different states. The goal of this method lies in a reduction in the muscle tension below the normal level on account of an improved body perception. Over time, the person should learn to bring about muscular relaxation whenever they want. Moreover, other signs of bodily unrest or excitation, such as palpitations, sweating or trembling, should be able to be reduced by relaxing the muscles. Moreover, muscle tensions can be found and loosened and hence pain states can be reduced. This method is preferably coupled to specific software and a suit. Music is preferably played to the user by way of headphones or any other acoustic output device, and the sensors in the suit carry out one and/or more muscle contractions in order to relax the user. A body journey can also be predetermined in an auditory fashion and the user receives haptic signals at the corresponding extremities in order to relax said user or school their bodily perception.

A further application example is that of sensors capturing the current state of the body during the EMS/EMG training and transmitting the ascertained information items to a unit. Suitable sensors can be selected from the following: BIA sensor, ultrasonic sensor, EMG sensor, EMS sensor, movement sensor, NIRS sensor, magnetoresistance sensor, moisture sensor, ECG sensor (in particular with an HRV measurement), elongation sensor (for measuring the respiratory rate), lactate sensor, temperature sensor, blood sugar sensor, and contact sensor. Thereupon, the data is transmitted to a computer unit in a wired and/or wireless manner, and the feedback to the muscle in order to train it is provided by EMS.

A further application example lies in sports, for training or massaging persons. Haptic sensors which adopt a massage function are installed into the suit. By way of example, a professional association football player has increased muscle tension after a game. The system according to the invention in the form of an apparel piece on the leg may, for example, loosen the thigh in order to improve an outflow of the blood in the leg. Initially, the thigh is loosened by way of a specific algorithm, in particular by way of vibrations or else mechanically. Then the whole leg is massaged upward, starting from the foot. The system may also be used for a whole body and/or partial body massage, wherein infrared sensors may be worked into the suit, said infrared sensors locally and/or globally heating an individual muscle group and/or a plurality of muscle groups.

If a person falls overboard and is exhausted, the system in the form of a suit can activate individual muscles or a plurality of muscles in order to produce heat. Alternatively or additionally, the system can monitor the vital functions in order to give the person time until they are saved.

A further embodiment relates to the use of the system by a user who is an astronaut. Here, the user can be trained in a targeted manner by way of the EMS training or it is possible to simulate sequences of movement. By way of example, if the astronaut has back pains, specific muscles can be trained. The training can be carried out according to prescriptions and individual training programs, with a control unit being connected to sensors on the astronaut. Provision can also be made for a GPS sensor which transmits the astronaut's height and position to the astronaut haptically.

A further application according to the invention is that of being able to instruct a user in the virtual space haptically to jump to the left, to the right, upward or downward in order to carry out all conceivable movements. Here, the user is guided by haptic feedback and/or the position of said user is captured by means of a GPS transmitter in order to localize them.

In a further embodiment, it is possible to iron on electrodes and also iron on the paths that conduct current and/or paths that do not conduct current. The electrodes to be ironed on may be present in the form of pre-manufactured PADs that can be ironed. The control unit can be fastened to the textile in a mobile and/or wired manner and said control unit cannot be ironed. The control unit serves to control the sensors. Preferably, a current supply can be connected to the system in any conceivable way in order to supply the system with power. The system can be controlled by way of a mobile terminal (smartphone) and/or by way of wires.

Electrodes may be worked into a textile as individual zones, either for the upper body or individual extremities and/or the lower body or individual extremities. The electrodes can consist of individual zones and/or a plurality of zones, which carry current or do not carry current.

An apparatus can be characterized in that sequences of movement can be trained by haptic feedback in conjunction with specific software. Preferably, a movement is demonstrated to the user in a virtual world and said user wears a suit which recognizes which muscles are active and which are not during the simulation in order to compare said muscles to the predetermined exercise (software). In particular, the user is assisted via EMS signals at the muscle groups that are important for the exercise. Preferably, a measurement in respect of which muscles are active is continuously carried out in the process. Hence, the user can learn any movement by this system in a virtual world, and constantly receive feedback.

An apparatus may further be characterized in that the electrodes consist of a material which transfers pulses onto the skin, said electrodes being combined with and/or folded, warp-knitted, embroidered or weft-knitted into a hydrophilic yarn. Alternatively, the electrodes may be provided with a moisture-providing layer that lies between the skin and conductor.

Moreover, the apparatus can be characterized in that the electrodes are manufactured from a conductive polymer; these silicone electrodes may be multilayered, i.e. consist of a conductive layer and/or a non-conductive layer which, preferably, is at most just as stretchable as the conductive silicone.

The apparatus can also be characterized in that the electrode consists of two outer ring electrodes and an inner ring electrode, or of an outer circle and an inner circle (the circles are respectively embodied as an electrode); these electrodes and/or electrodes are suitable for bipolar and/or unipolar currents.

The apparatus can be characterized in that a ratio for adapting at least two stimulation pulse parameters is predetermined in the data processing device and, if there is a change in measurement values from one or more sensors, the adaptation of these parameters is provided in accordance with this ratio, wherein the stimulation pulse parameters may be parameters for the same electrode or different electrodes.

Moreover, the apparatus can be characterized in that one or more sensors are configured to record different measurement values, wherein, in particular, the measurement principle of these sensors is based on different physical principles and the data processing unit (4) is configured to weight these measurement values in a comparison and trigger stimulation pulses herefrom and, in the process, modify stimulation pulse parameters.

### BRIEF DESCRIPTION OF THE FIGURES

The Figures, to which the following exemplary description relates, are described below. In detail:

FIG. 1 shows a schematic diagram of the apparel with essential functional elements that are involved in the method according to the invention, and a sensor;

FIG. 2 shows a selection of possible apparel pieces and attachment options for sensors;

FIG. 3 shows an electrode which has a concave form;

FIG. 4 shows a suit with a massage function;

FIG. 5 shows a suit and the possible training function thereof;

FIG. 6 shows a feedback method in a virtual world;

FIG. 7 shows a pair of trousers with a haptic application;

FIG. 8 shows a screen with sensors that are fastened to different points of the suit,

FIG. 9 shows a screen with various program options,

FIG. 10 shows a screen with various adjustment modifications,

FIG. 11 shows an adjustment screen,

FIG. 12 shows a screen with a sports-specific exercise, and

FIG. 13 shows an access screen to a virtual fitness studio course offering, and

FIG. 14 shows a schematic representation of a controller of stimulated pulses.

### DETAILED DESCRIPTION OF THE INVENTION

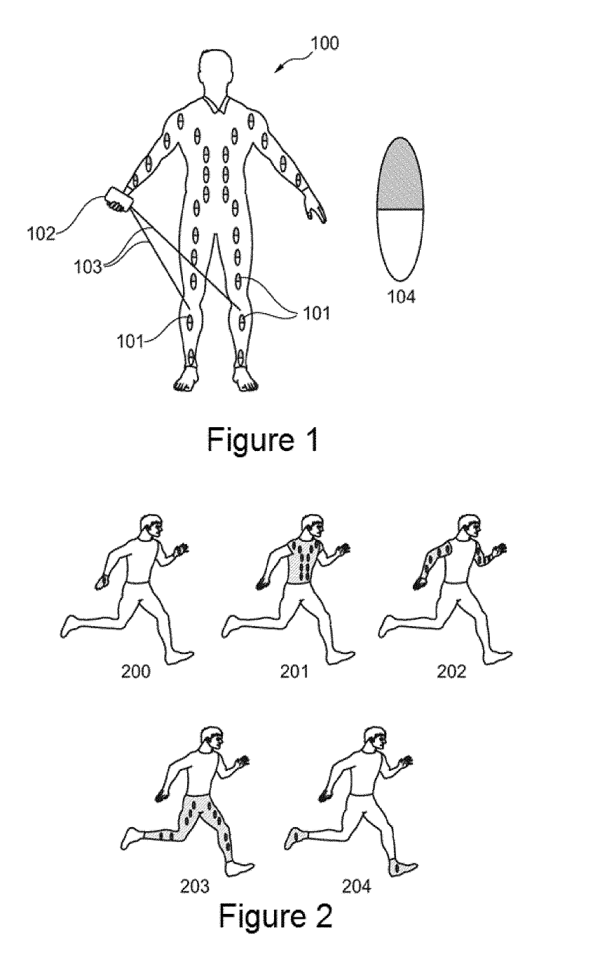

FIG. 1 shows, by way of a schematic diagram, a possible suit with the essential functional elements that are involved in the method according to the invention and the apparatus. 100 is used to denote a suit, on which a multiplicity of sensors or electrodes 101 are fastened, the latter both measuring EMG signals of a body and transferring EMS signals to a body. The dual function of the electrodes/sensors is elucidated by the two-tone representation.

Provision can be made of individual and/or a plurality of electrodes 101. 102 is used to denote a mobile terminal, which can preferably receive and/or transmit signals 103. It may be a mobile smartphone and/or a stationary unit. 104 is used to denote a schematic diagram of a sensor/electrode, which captures sensor data from a body (EMG signal) or transfer a signal (EMS signal) to a body. These may also be any other conceivable sensors which capture biological and/or physical data from humans. Many further embodiments are conceivable by way of different shaping, material selection, type, and positioning of the sensors and the processing. In FIG. 1, it is possible to see a user interacting with the system. In FIG. 1, the system is configured as a suit, into which the sensors are sewn in the form of a yarn. Moreover, this embodiment that is sewn into a suit or else any other apparel piece is possible by way of electroactive and/or electrosensitive yarn. The system is supplied with power by means of a current-supplying cable (not depicted). Alternatively, there may also be a current generator present, the latter producing current from kinetic energy, said current then being stored in a battery. A possible embodiment could include wireless inductive charging. Piezoelectric plastic (nano-generator) could be a further conceivable embodiment.

The apparel pieces presented in FIG. 2 by 200, 201, 202, 203, and 204 only represent a selection of many further conceivable apparel pieces. One or more of the following sensors may be integrated into the system: BIA sensor, ultrasonic sensor, EMG sensor, EMS sensor, movement sensor, NIRS sensor, magnetoresistance sensor, moisture sensor, ECG sensor (including HRV measurement), elongation sensor (respiratory rate), lactate sensor, temperature sensor, blood sugar sensor, pulse sensor, and contact sensor. All sensors may be worked into the textile or apparel piece and/or into a control device that may be fastened to the apparel.

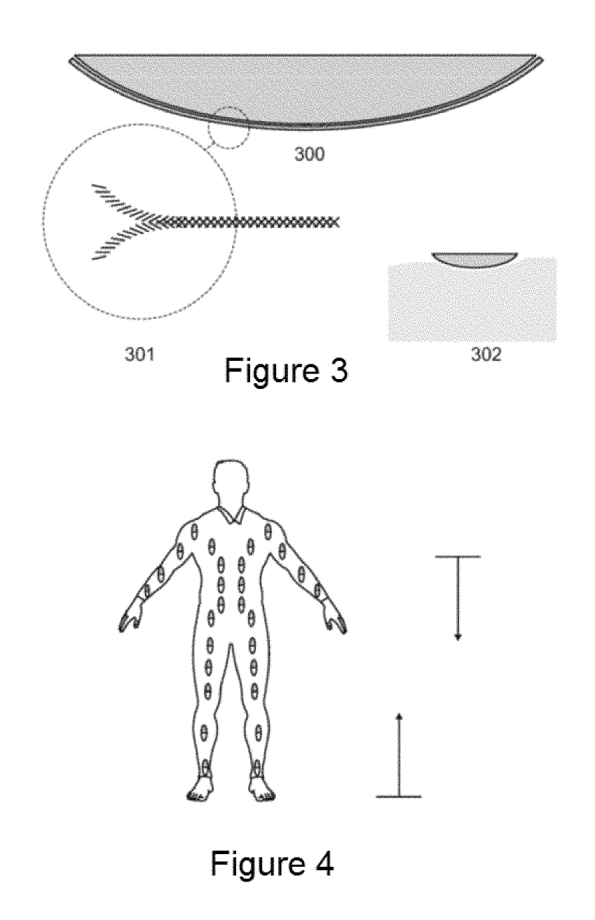

The schematic diagram of an electrode presented in FIG. 3 is concave in this exemplary embodiment 300. Many further embodiments are conceivable by way of different shaping, material selection, type, and position of the electrode and processing. A schematic diagram is depicted in the section of 301, in which it is possible to see a textile with a hydrophilic silicone yarn. 302 is used to denote that a better contact with the body arises as a result of this form. A concave electrode can preferably be used in concave body regions, such as between the breasts or in the region of the armpits.

The schematic diagram presented in FIG. 4 illustrates the option of a haptic massage method, which is integrated into a suit and transfers (electrotactile, mechanotactile or vibrotactile stimuli) by haptic sensors. A suit which can be used for a whole body massage can be seen. Any type of stimuli provision is conceivable, e.g. rising, falling, pulsating, vibrating, tapping and wave-shaped signals (partly symbolized by arrows). The method can be integrated into any apparel piece which is in contact with the body. Long or short socks which may be worn during a flight and which are able to transfer, from bottom to top, a pulsating vibration signal over the entire area and/or else an EMS signal, in order to activate the muscle groups of the lower extremities, are conceivable. Shorts which activate and/or stimulate the outer skin are within the scope of the invention. Any type of signal guide is conceivable. It is also possible to work sensors for capturing the vital parameters into the textile. Haptic stimulation can preferably be a mechanical stimulation such as e.g. by vibrating units. This may also be a thermal stimulation. Short pulses may be used in the case of an electrostimulation.

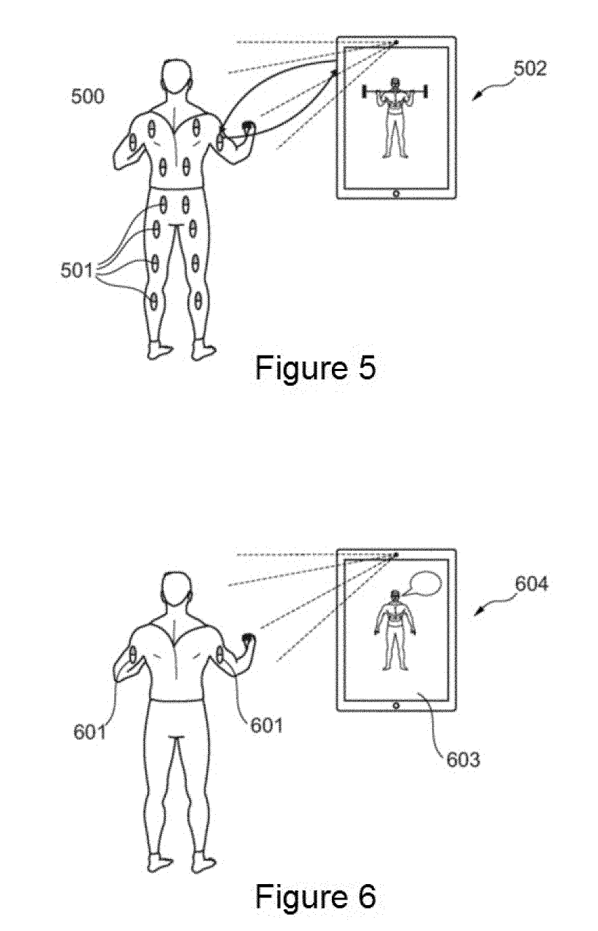

The schematic diagram presented in FIG. 5 shows a therapy or training method according to the invention. 500 is used to denote a suit with sensors 501, said suit being able to receive and/or transmit signals (symbolized by arrows). In the case of tension and/or increased muscle activity, the sensors are able to measure the activity and evaluate this by way of analysis software. If a muscle being too active is disclosed during the analysis, the muscle is activated on the contralateral side in order to trigger an inhibition, as a result of which the muscle loses its tone and/or relaxes. The method operates according to the principle of afferent collateral inhibition. The principle of afferent collateral inhibition is described below: muscular work (muscle contraction) is only possible if the antagonist is inactivated at the same time as the activation of the agonist, and vice versa. This is achieved by interconnecting afferences and efferences in the spinal column by way of inhibitory interneurons. FIG. 5 represents the reception of the sensor data which receives the activity signals from the muscles, and transmits these to a controller, such as e.g. a mobile terminal (smartphone, tablet PC). An analysis software method runs on the mobile terminal 502. The data are transferred in a mobile and/or wired manner. 501 is used to denote the sensors which capture the muscle activity and transmit this to the mobile terminal. The measured data are not necessarily transmitted directly by the sensors; instead, the sensors may be connected to a data transfer unit which undertakes the transfer. 501 is used to denote the sensors/electrodes which transfer the muscle-stimulating stimuli to the skin. These are transferred from the mobile terminal 502 and/or in a wired manner. 502 is used to denote the software, represented by a trainer method.

The schematic diagram presented in FIG. 6 shows a user with a system according to the invention in the form of an apparel piece that is worn on the upper body and a visualization unit in the form of a screen 604 (by way of example, goggles, in particular 3D goggles, are also conceivable). The user interacts with a virtual world (with virtual surroundings). By way of the visualization unit 604, it is possible to see a virtual trainer 603, who demonstrates an exercise and provides instructions. The person exercising repeats this exercise. The trainer provides a training instruction which the user should simulate. If they do not carry out the exercise correctly, this is captured by way of a sensor and the software processes the signal and transmits a haptic signal (electrotactile, vibrotactile or mechanotactile) to the user. This signal may be an EMS signal which is configured to immediately bring about a muscle activation. Alternatively, it is possible to provide a signal at a frequency that is unsuitable for muscle activation. This signal is sensitively recognized by the body and the user can subsequently deliberately carry out a corrected movement. 603 is used to denote a portion of the visualization unit 603 which provides the user with an instruction to carry out the movement correctly while the system regulates the performance of the movement by way of the sensors 601. By way of the sensors 601 (e.g. strain gauges) in the textile, the system recognizes whether the movement was carried out correctly. If the movement was not carried out correctly, an avatar shows how the exercise is done correctly in real time. Hence, a realistic recognition of the exercise is possible. By way of this virtual feedback method (by means of goggles or a helmet, visor, contact lens, display that is situated in front of the eyes), any conceivable movement can be learned and a new interaction is also possible. FIG. 6 shows an apparel piece, into which individual sensors or a plurality of sensors 601 have been worked, said sensors being able to transmit and/or receive signals. Measured values can be transmitted by way of a transmission module (e.g. radio, Bluetooth) that is connected to the sensor. By way of this, it is also possible to receive data, such as e.g. activation information items for individual electrodes. It is also possible to capture vital parameters, as described above. EMS signals can also be transferred from the virtual trainer 603. From a technical point of view, there are a number of options for measuring movement (e.g. acceleration sensors, sports biomechanics). Use is often made of miniaturized piezoelectric acceleration sensors that are made from silicon and convert the pressure variations caused by an acceleration into electrical signals. Small, robust sensors have a mass of only a few grams and a high sensitivity with a good resolution of the signal. Relatively new piezoresistive and piezocapacitive sensors supply a signal which shows not only the acceleration but also the inclination of the sensor (positioning in relation to gravitational acceleration). The DC voltage components of the signal differ in the case of horizontal or vertical positioning, and consequently it is also possible to determine the position of the body in space. Gyrosensors can also measure the angular acceleration. An acceleration sensor reacts with maximum sensitivity only in one dimension, and so two or three sensors have to be combined in order to be able to capture movements in the plane or in three-dimensional space. Measurements in one or two dimensions (axes) suffice for many purposes, while the human movement behavior should be measured in the three spatial dimensions (planes). The attached sketch only serves for illustration purposes and only constitutes one of many possible embodiment variants.

In one exemplary embodiment, a sensor, in particular a strain gauge, may be configured to recognize a posture, such as in particular the angular position of a joint, of a person training with the system or to recognize a movement of a body part or of the entire body of the person training and to bring about electrostimulation depending on the posture, in particular the angular position, or the movement, in particular the speed thereof.

The object of a preferred method is to select a training course in a virtual sports studio. A suit, as described above, which renders it possible to receive haptic signals lies within the scope of the invention. Preferably, the user is provided with the option of choosing a virtual course by means of a visualization unit. The selection method can be brought about by way of a gesture of the user or by way of a targeted movement to the respective course. The gestures are recognized by way of the apparel piece, in particular the suit, and forwarded to the controller. The controller activates the desired function or the desired program. The system may comprise a user interface with a sensor which, in particular, may be a camera, an ultrasonic sensor or a radar sensor, and/or the user interface can be adapted to control the EMS system and/or individual pulse parameters by gestures. By way of example, the visualization unit can show the user a direction. It is possible to navigate the user and let them carry out jumps to the right, to the left, to the front, to the back or into the air. The virtual trainer provides said user with instructions to move. The system can also be used for learning or online schooling.

If a user performs a movement that was not carried out correctly, this is recognized by the virtual trainer and the latter demonstrates the exercise in a precise manner and provides instructions to said user to the effect of optimizing their movements. The virtual trainer also simulates the movements and provides optimization instructions for carrying out movements. Hence, the trainer is also able to teach the user a sports-specific exercise, such as e.g. a golf swing and all conceivable movement embodiments. It is also possible to complete a specific online-supported EMS training with a virtual trainer. It is also within the scope of the invention to provide the user with a mirror function on the visualization unit so that said user can orient themselves visually. The method recognizes the execution of the movement, compares it in the software and provides a correction instruction by the virtual trainer.

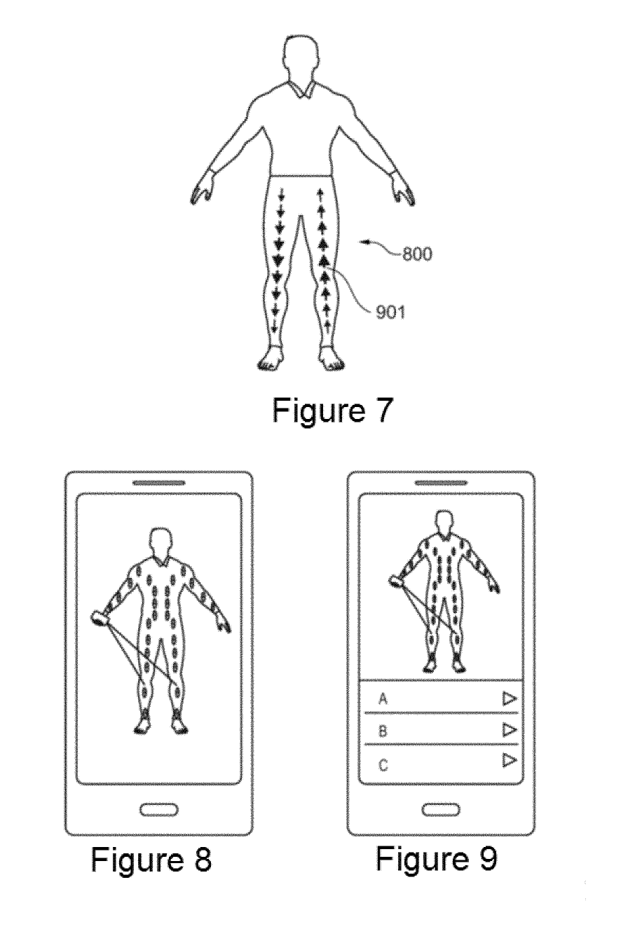

The schematic diagram presented in FIG. 7 shows a massage application for use e.g. in professional sports. The goal of the example described here is to present a massage method for professional sports. An example of the lower extremities 900 is presented. These trousers represent any conceivable apparel piece in an exemplary manner. In order to ensure a return flow of the liquid collected in the lower extremities after sports, the thigh is worked on first (by haptic means) on the thigh by way of a pulsating function. Then, the massage is continued from bottom to top 901. This is because, after the thigh, it is possible to improve the return flow and to carry out a massage and/or therapy from below (caudal to cranial). The massage forms can be preparation massages, relaxation massages or activation massages.

A PMR (progressive muscle relaxation) method which transfers the stimuli in the suit by way of haptic sensors, i.e., in particular, actuators such as electrodes, also lies within the scope of the invention. The progressive muscle relaxation method according to Edmund Jacobson is a method in which a state of deep relaxation of the entire body should be obtained by deliberate and conscious relaxation and/or tensioning of specific muscle groups. Here, the individual muscle groups are initially tensioned successively in a specific sequence, the muscle tension is briefly held and the tension is subsequently released. Here, the concentration of the person is directed to the change between tension and relaxation and the sensations that accompany these different states. The goal of this method lies in a reduction in the muscle tension below the normal level on account of an improved body perception. Over time, the person should learn to bring about muscular relaxation whenever they want. Moreover, other signs of bodily unrest or excitation, such as palpitations, sweating or trembling, should be able to be reduced by relaxing the muscles. Moreover, muscle tensions can be found and loosened and hence pain states can be reduced. By way of example, it is possible to trigger a haptic signal at the foot, said signal signaling to the user which muscle in the body should be tensioned and when this muscle should be relaxed again. This method is possible using a suit which transfers vibrotactile, electrotactile or mechanotactile stimuli by way of haptic sensors. In particular, this signal can be different to an EMS signal. The difference lies in the frequencies of the activation. It is possible to activate individual sensors/electrodes and/or a plurality of sensors/electrodes; all applications can be transmitted in a wireless and/or wired manner to a controller, e.g. a mobile terminal (smartphone) and/or be received from there. Optionally, it is also possible to transmit relaxing music.

A suit in which the sensors/electrodes or actuators also transfer haptic signals also lies within the scope of the invention. The transfer can take place by way of a closed water circulation system and/or via a closed air system. A suit can thus consist of two different zones. One zone lies on the body and the outer serves for delimiting the surroundings. Nozzles which transfer a haptic signal to the skin via air pressure or water pressure are between the two zones in order to carry out one of the above-described methods.

A suit within the scope of the invention with a diagnostic function can work online and/or offline. This also applies to all methods described above. A suit which has one or more vital sensors also lies within the scope of the invention. It is possible to measure any biodata and transmit these to the controller or the control device in a mobile manner and/or via a cabled connection. It is possible to provide sensors which capture the temperature and all conceivable vital parameters and transfer these in a wired and/or wireless manner to the controller or the mobile terminal (a smartphone). The data can be evaluated by an online medical practitioner or by diagnostic software in order to transmit health suggestions to the user. By way of example: visiting a medical practitioner is recommended if the temperature is too high. Moreover a screen of the controller, which, via 3D goggles, a screen or any conceivable indicating device, indicates the user's health state to the user, also lies within the scope of the invention. This may be physiological or else anatomical and comprise any conceivable visualization.

The screen presented in FIG. 8 shows the suit which can activate individual sensors by touching the screens. The suit may facilitate the local or global use of electrodes for one of the methods described above. This control can be mobile or wired. The function can be online and/or offline.

The screen presented in FIG. 9 shows a function screen which provides the option of selecting individual programs, as described above.

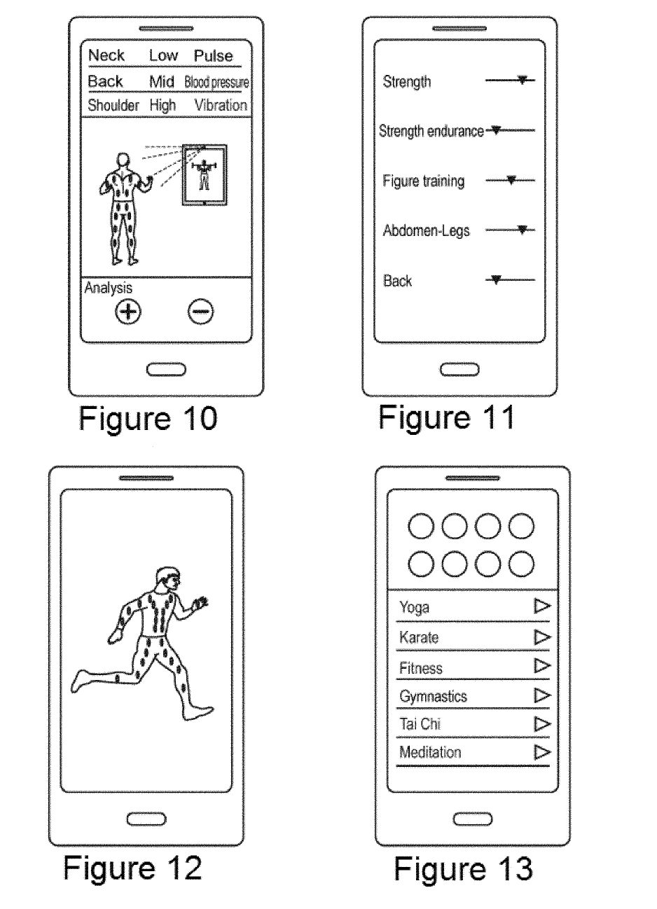

The screen presented in FIG. 10 shows the control of the therapy method and/or training method, which is able to capture every body region and be set individually.

The screen presented in FIG. 11 shows various setting modes.

The screen presented in FIG. 12 shows a sports-specific exercise, which can be learned as described above. The athlete is prescribed an exercise and must then simulate it. If it is not carried out correctly, the athlete is assisted by the system and they receive a stimulus via an EMS signal in order to activate the muscle groups which should be used. The stimulus can be transmitted by any haptic sense (vibrotactile, electrotactile or mechanotactile stimuli). The system also recognizes what muscles are active. Hence, the athlete is able to learn any movement or optimize it. Any sport and/or movement is possible. By way of example, the user can learn a golf swing. Additionally, any haptic signal can be used for communicating a stimulus to the user. In this method of learning movements, it is possible to recognize movements and/or transmit movement data. The method runs by way of control software which compares the movement to the predetermined movement and optimizes the movement via muscle activity measurements and/or muscle activations.

The screen presented in FIG. 13 shows the access to an online sports studio, which can be used offline and/or online. By tapping one of the buttons in the upper region of the screen, the user can access a course or undertake individual settings.

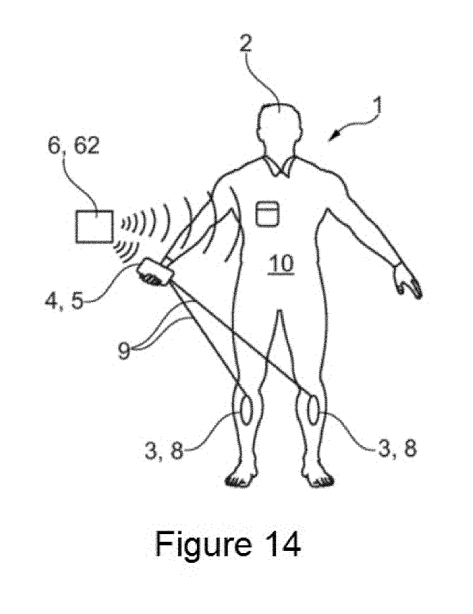

FIG. 14 shows a schematic representation of a controller of stimulation pulses. The system 1 for controlling stimulation pulses during a stimulation on a user 2 comprises at least a sensor 3, a data processing unit 4, and a pulse unit 5. In the embodiment presented in FIG. 14, the electrodes 8 and the sensors 3 are connected to a textile, a track suit 10 in this case, and respectively securely connected in a lower leg region of the track suit 10. As a result, a wearable system 1 is provided, which allows the user to carry out the stimulation application in a manner that is unimpeded in space and/or in terms of their freedom of movement. Here, the sensor 3 is e.g. suitable for measuring a measurement value, in particular the EMG activity of the user 2. This advantageously allows measuring EMG activity of the user 2 and triggering a stimulation pulse, in particular an EMS pulse, which, depending on the measurement value or control signal, has been modified in terms of one or more stimulation pulse parameters. Advantageously, one or more sensors 3 of the same type or of different types can be arranged in the system 1.

The data processing unit 4 is configured to compare the measurement value to a threshold and generate a control signal for the pulse unit 5 if the measurement value and the threshold have a predefinable relationship to one another. In the embodiment shown in the present case, the pulse unit 5 and the data processing unit 4 are attached in a common casing, which can be carried by the user 2 in one hand or, optionally, be placed into a pocket or be detachably connected to the track suit 10. Here, the pulse unit 5 is suitable for triggering stimulation pulses and configured to modify one or more stimulation pulse parameters depending on the control signal.

A method that is likewise within the scope of the invention, in which a pulse unit triggers one or more stimulation pulses, comprises at least the following steps: a) measuring a measurement value, b) comparing the measurement value to a threshold, c) generating a control signal if the measurement value and the threshold have a predeterminable relationship to one another, and d) modifying a stimulation pulse parameter depending on the control signal.

Here, the measurement value that is measured by means of sensors is compared to a threshold by means of suitable algorithms. Such an algorithm can advantageously be predetermined or adjustable or predeterminable in the data processing unit. If it is determined that the measurement value and the threshold have a predefined relationship to one another, an appropriate control signal is generated and a pulse parameter is modified depending on the control signal. A corresponding stimulation pulse with a modified pulse parameter can then be triggered by the pulse unit. Hence, e.g. the stimulation pulse intensity can be increased or reduced, depending on the measurement value. Likewise, it is alternatively or additionally possible to modify further stimulation pulse parameters such as pulse type, intensity, duration of the stimulation pulse, frequency, ramp, pulse pause, individual pulse width, and/or individual pulse duration.

The system 1 presented in FIG. 14 moreover comprises a user interface 6 with an input means 62, e.g. buttons. In the presented embodiment, the user interface 6 is arranged in a casing that is separate from the data processing unit 4 and the pulse unit 5 and configured as a remote control. Consequently, it is possible to control and set the data processing unit 4 and the pulse unit 5 by means of the remote control that comprises the user interface 6, without the user 2 having to carry the remote control during the stimulation application. The portable casing comprising the data processing unit 4 and the pulse unit 5 further comprises an energy source 7.

Provision can be made of feedback means which provide information about the next EMS pulse. By way of example, an EMS pulse can have a duration of 3 seconds and this can be followed by a pause of, for example, 3 seconds. So that the user is not surprised by the next pulse, e.g. an optical signal may be output. By way of example, this may occur on a back-of-the-hand unit. An electronic component, on particular a communication module, may be fastened to the back of the hand using fastening means. Alternatively, an armband can also be used to this end.

By way of example, an LED can light up or blink for a second or half a second before the start of an EMS pulse. Haptic feedback is also possible. Thus a vibration can be exerted by means of a corresponding communication module. The hand is very sensitive and thus it is possible to perceive such vibrations well. In addition to the aforementioned output means, i.e. means that supply the user with information items about the system state, provision can be made of input means. Parameters of the stimulation, such as pulse intensity, frequency, signal type (rectangular or sinusoidal) can be selected by way of individual buttons or a button field (or touchscreen). It is also possible to select and activate individual electrodes (or groups of electrodes) of the EMS. In the aforementioned examples, the communication module is preferably fastened to the hand or wrist, in particular to the back of the hand. In addition, a communication module can be fastened to a different point on the apparel that is used during EMS training. By way of example, a communication module can be fastened to the nape. A feedback module arranged there will preferably output haptic signals or acoustic signals as these can be perceived well on the neck. Electric signals can also be used as feedback for the restart of the stimulation. In this case, use is preferably made of a frequency range that is not suitable for the stimulation. For EMS, frequencies of 20 to 300 Hz are preferably used in some applications. Hence, the feedback signal can be a DC signal or a low-frequency signal <20 Hz or greater than 1 kHz.

For the communication module aspect of the invention, the following configurations are conceivable:

Electrostimulation device comprising at least one apparel piece, which comprises a multiplicity of electrodes for electrostimulation, an energy source for electrostimulation, such as, in particular, a battery or an accumulator, which is connected to the apparel piece; wherein the EMS device further comprises a feedback apparatus that is configured to be worn on the body of a person training with the EMS device, and a controller is configured to cause electrostimulation signals and, further, transmit a signal to the feedback apparatus at a defined period of time prior to an electrostimulation signal.

Electrostimulation device, configured to receive EMG signals and/or for transmitting EMS signals to a human body in order to train it.

Electrostimulation device, characterized in that the feedback apparatus is connected to an apparel piece, in particular the apparel piece comprising the electrodes and energy source.

Electrostimulation device, characterized in that the feedback device is configured to emit an optical signal and attachable to the wrist or the hand of a person training and, in particular, the feedback device is attachable to the back of the hand of the person training.

Electrostimulation device, characterized in that the feedback device is configured to emit an optical signal and arranged in goggles, a helmet, a visor, a contact lens, a display that is situated in front of the eyes.

The embodiments and features presented in this description can be combined freely with one another.

1. A portable apparatus for transmitting electrical muscle stimulation (EMS) signals to a human body in order to train said body and for controlling stimulation pulses during a stimulation on a user, comprising: at least a sensor, at least a data processing unit, at least a pulse unit, and one or more electrodes for electrical muscle stimulation, wherein
a) the sensor is suitable for measuring one or more measurement values,
b) the data processing unit is configured to generate a control signal for the pulse unit depending on the measurement value(s) from the sensor or sensors,
c) the pulse unit is suitable for triggering EMS pulses and configured to modify one or more EMS pulse parameters depending on the control signal,
wherein a comparison between the measurement value(s) and a threshold is carried out
wherein the electrodes, the sensor, the data processing unit or the pulse unit, or all four of them, are fastened to an apparel piece, and
wherein the apparatus further comprises a visualization unit for presenting a virtual reality.

2. (canceled)

3. The apparatus as claimed in claim 1, wherein the apparatus is embodied to be worn on the body of a user in the form of an apparel piece and configured to receive or transmit wireless or wired signals, or both, of the body at least from one or more sensors, and that the apparatus is embodied to be worn on the body, and comprising fastening means which place the apparatus directly on a body part.

4. The apparatus as claimed in claim 1, wherein at least one sensor is configured to read electromyography signals.

5. The apparatus as claimed in claim 1, wherein at least one electrode is configured to capture measurement data about the state of the user or transmit sensor data, or both, to a human body, and wireless communication is provided.

6. The apparatus as claimed in claim 1, wherein the apparatus is a textile or a constituent part of a textile, or one or more sensors, or both, for capturing one or more vital parameters are integrated into the textile or fastened or fastenable, or both, to the textile, and wherein the sensors comprise a BIA sensor, movement sensor, NIRS sensor, magnetoresistance sensor, moisture sensor, ECG sensor, HRV sensor, strain gauges, spiroergometry sensor, lactate sensor, temperature sensor, contact sensor, or a and blood sugar sensor.

7. The apparatus as claimed in claim 1, wherein the pulse unit is connected to a user interface in a wireless or wired manner, or both, said user interface elucidating the stimulation on the hand or the wrist within the meaning of a visualization unit, signaling the change between pulse and rest, wherein said visualization unit can provide acoustic or visual or haptic signals, or all three signals, by vibrating, and, at the same time, be used to control the intensity within the meaning of an input means or indicates signals by way of LEDs or both; and the user interface can be fastened, to the thumb by way of a wristband which is precisely not a wristband but can be worn over the back of the hand.

8. (canceled)

9. The apparatus as claimed in claim 1, wherein for the purposes of training a person in a virtual world (environment), use is made of an apparel piece, which is equipped with one or more haptic sensors, and 3D goggles, helmet, visor, contact lens, or a display situated in front of the eyes, and a virtual interface, wherein haptic sensors are integrated into the apparel piece, said sensors measuring the position in space and, with software assistance, comparing said position to that of the virtual trainer in order to recognize wrong movements and indicate these in the virtual environment by auditory or optical signals, or both, or wherein an avatar displays or performs an improved execution of movements or simulates, or both, the wrong movements in order, then, to clarify these by an improved execution of movements.

10. The apparatus as claimed in claim 1, wherein sequences of movements are recognizable by haptic feedback in conjunction with specific software and a movement is presentable to the user in a virtual world, and an apparel piece is configured to identify which muscles are active and which are not in order to compare this to the predetermined exercise (software), and, wherein in particular, the user obtains support for the groups of muscles that are important to the exercise by way of electrical muscle stimulation signals, and there is constant measurement of which muscles are active.

11. (canceled)

12. (canceled)

13. (canceled)

14. (canceled)

15. (canceled)

16. The apparatus as claimed in claim 1, wherein the visualization unit for presenting a virtual reality, is configured to present a 2D view or a 3D view of the virtual reality or the visualization unit comprises a display, or a screen, or data goggles, or a helmet, or a visor or a contact lens; or both.

17. The apparatus as claimed in claim 1, wherein a visualization unit is configured to produce an image of the person training, wherein the image may be changeable in terms of size, look, and apparel by way of the system or entries into the system.

18. (canceled)

19. (canceled)

20. The apparatus as claimed in claim 1, wherein, for the image of the person training, one or more defined sequences of movement are stored, and the system is configured to assist or correct by way of electrostimulation the movement of the person training in such a way that the deviation between a performed movement by the person training and the defined sequence of movements is minimized.

21. (canceled)

22. A method for controlling stimulation pulses during stimulation on a user using a system as claimed in claim 1, wherein a pulse unit triggers one or more stimulation pulses, said method comprising the following steps:
a. measuring a measurement value,
b. comparing the measurement value to a threshold,
c. generating a control signal if the measurement value and the threshold have a predeterminable relationship to one another, and
d. modifying a stimulation pulse parameter depending on the control signal.

23. The method of claim 22, wherein the stimulation pulse parameter is selected from any of a pulse type, intensity, duration of the stimulation pulse, frequency, ramp, pulse pause, individual pulse width, individual pulse duration, the rise time, or fall time.

24. (canceled)

25. The method of claim 22, wherein steps a to d are repeated at least every 10 minutes during the duration of an application.

26. The method of claim 25, wherein a visualization unit is provided, which presents a virtual reality.

27. (canceled)

28. (canceled)

29. (canceled)
